(19) United States
(12) Patent Application Publication
(10) Pub. No.: US 2023/0374591 A1
(43) Pub. Date: Nov. 23, 2023

(54) METHODS AND AGENTS FOR TREATING, PREVENTING, DIAGNOSING, AND EVALUATING THERAPY FOR FIBROTIC, AUTOIMMUNE, AND INFLAMMATORY CONDITIONS

(71) Applicant: THE TRUSTEES OF DARTMOUTH COLLEGE, HANOVER, NH (US)

(72) Inventors: Yue WANG, Arlington, MA (US); Michael L. WHITFIELD, Etna, NH (US)

(21) Appl. No.: 18/030,801

(22) PCT Filed: Oct. 7, 2021
(86) PCT No.: PCT/US2021/053880

§ 371 Date: Apr. 7, 2023

Related U.S. Application Data
(60) Provisional application No. 63/089,207, filed on Oct. 8, 2020.

(51) Int. Cl. C12Q 1/6883; A61K 45/06; A61K 35/28

(52) U.S. Cl. CPC C12Q 1/6883; A61K 45/06; A61K 35/28; C12Q 2600/158; C12Q 2600/106; C12Q 2600/136

(57) ABSTRACT

Novel methods of treating and/or preventing a fibrotic, autoimmune, and/or inflammatory disease or condition such as scleroderma, methods of diagnosing a fibrotic, autoimmune, and/or inflammatory disease or condition such as scleroderma and methods for predicting whether a therapy for a fibrotic, autoimmune, and/or inflammatory disease or condition such as scleroderma is effective are provided which involve detecting and/or modulating the expression of function of specific genes correlated to a fibrotic, autoimmune, and/or inflammatory disease prognosis. Also, agents and compositions containing for treating or preventing a fibrotic, autoimmune, and/or inflammatory disease or condition such as scleroderma and kits for detecting genes associated with a fibrotic, autoimmune, and/or inflammatory disease or condition such as scleroderma are provided.

### CROSS-REFERENCE TO RELATED APPLICATIONS

This application claims the benefit of priority to U.S. Provisional Appl. No. 63/089,207, filed Oct. 8, 2020, of which is hereby incorporated by reference in its entirety.

### SEQUENCE DISCLOSURE

This application includes as part of its disclosure an electronic sequence listing text file named “1143252o004801.txt”, having a size of 15,215 bytes and created on Sep. 28, 2021, which is hereby incorporated by reference in its entirety.

### FIELD OF THE INVENTION

The present invention disclosed herein relates to methods of treating and/or preventing a fibrotic, autoimmune, and/or inflammatory disease or condition, methods of diagnosing scleroderma, methods of diagnosing a subject with a fibrotic, autoimmune, and/or inflammatory disease or condition, methods of determining whether a therapy for a fibrotic, autoimmune, and/or inflammatory disease or condition is effective in a subject, methods of screening for a therapeutic agent for a fibrotic, autoimmune, and/or inflammatory disease or condition, methods of predicting whether a patient with a fibrotic, autoimmune, and/or inflammatory disease or condition will respond to hematopoietic stem cell transplant (HSCT), methods of predicting whether a patient with a fibrotic, autoimmune, and/or inflammatory disease or condition will respond to cyclophosphamide (CYC), and methods of determining whether a patient with a fibrotic, autoimmune, and/or inflammatory disease or condition should receive HSCT or CYC. The present invention further relates to agents for treating or preventing a fibrotic, autoimmune, and/or inflammatory disease or condition, antibodies and antigen-binding antibody fragments for treating or preventing a fibrotic, autoimmune, and/or inflammatory disease or condition, and kits for detecting expression of genes associated with a fibrotic, autoimmune, and/or inflammatory disease or condition.

### BACKGROUND OF THE INVENTION

Fibrosis is formation or deposition of fibrous connective tissue, characterized by excess accumulation of extracellular matrix (ECM) such as collagen, in an organ or tissue and can severely disturb the function of such an organ or tissue. Various autoimmune diseases and inflammatory diseases are known to cause fibrosis, and currently there is no therapy that reverses or cures such fibrosis.

Systemic Sclerosis (SSc; scleroderma) is a complex and rare autoimmune disease with unclear etiology (Allanore Y. et al., Nat Rev Dis Primers. 2015 Apr. 23; 1: 15002. doi: 10.1038/nrdp.2015.2. [PMID: 27189141]). Patients have vascular damage, skin fibrosis, and internal organ dysfunction that includes the gastrointestinal tract system, lungs, heart and kidneys. Based on the gene expression signatures, Applicant previously identified molecular “intrinsic” subsets (fibroproliferative, inflammatory, limited, and normal-like) in multiple SSc cohorts (Milano A. et al., PLoS One. 2008 Jul. 16; 3(7):e2696. doi: 10.1371/journal.pone.0002696. [PMID: 18648520]; Pendergrass S. A. et al., J Invest Dermatol. 2012 May; 132(5): 1363-73. doi: 10.1038/jid.2011.472. Epub 2012 Feb. 9. [PMID: 22318389]; and US20110190156A1). Applicant further discovered that different intrinsic subsets had distinct responses to different therapies (Hinchcliff M. et al., J Invest Dermatol. 2013 August; 133(8): 1979-89. doi: 10.1038/jid.2013.130. Epub 2013 Mar. 14. [PMID: 23677167]; Chakravarty E. F. et al., Arthritis Res Ther. 2015 Jun. 13; 17(1):159. doi: 10.1186/s13075-015-0669-3. [PMID: 26071192]; Gordon J. K. et al., Arthritis Rheumatol. 2018 February; 70(2):308-316. doi: 10.1002/art.40358. Epub 2017 Dec. 29. [PMID: 29073351]; and WO2012118856A1).

Currently, no medication cures SSc, and the focus of SSc treatment is slowing disease progression and managing specific symptoms. For managing Raynaud's phenomenon, calcium channel blockers (e.g., nifedipine, amlodipine, diltiazem, felodipine), PDE5 inhibitors (e.g., sildenafil, tadalafil, vardenafil), endothelin receptor antagonists (e.g., bosentan, macitentan), Angiotensin II receptor antagonists (e.g., losartan, valsartan, olmesartan), prostacyclin analogs (e.g., iloprost, epoprostenol, treprostinil), and/or topical nitroglycerine (e.g., MQX-503) may be prescribed. For skin fibrosis, D-penicillamine and methotrexate may be used but these drugs have limited effectiveness in addressing skin thickening and have serious side effects. Steroids (e.g. prednisone) are not sufficiently effective in most SSc subtypes but may be effective in SSc with arthritic symptoms. Nonsteroidal anti-inflammatory drugs (NSAIDs) such as COX-2 inhibitors may be used to alleviate muscle and join pain. For pulmonary complications such as interstitial lung disease (ILD) and pulmonary artery hypertension (PAH), immunosuppressants are often helpful. Recently approved nintedanib (triple tyrosine kinase inhibitor) addresses ILD, and acitentan (endothelin receptor antagonist) and riociguat (guanylate cyclase stimulator) address PAH by relaxing blood vessels. For SSc-associated kidney diseases, ACE inhibitors (e.g., captopril, enalapril) may be used. Hematopoietic stem cell transplant (HSCT) and therapeutic plasma exchange (TPE) seem to be effective, but the high cost, the invasiveness of the procedures, the use of an immunosuppressant (in case of HSCT), and the need of permanent and regular treatment (in case of TPE) create various risks and difficulties for the patients. A therapeutic method that stops or reverses overall progression of SSc is still in need.

The pathology of SSc is complex and remains incompletely understood, but the field agrees that immune dysfunction is one of the most important components of the pathogenesis (Frantz C. et al., Front Immunol. 2018; 9: 2356. Published online 2018 Oct. 15. doi: 10.3389/fimmu.2018.02356 [PMID: 30374354]). Therefore, reversing immune dysfunction may be a key in treating and potentially curing SSc. The majority of the studies reported decreased frequencies and/or impaired function of regulatory T cells (Tregs) in SSc (Kalekar L. A. et al., Int Immunol. 2019 Jul. 13; 31(7): 457-463. doi: 10.1093/intimm/dxz020. [PMID: 30865268]). Also, some SSc patients who received hematopoietic stem cell transfer (HSCT), demonstrated enhanced Treg engraftment and increased Treg numbers which correlated with reduced skin fibrosis (Arruda L. C. M. et al., Blood Adv. 2018 Jan. 23; 2(2): 126-141. doi: 10.1182/bloodadvances.2017011072. [PMID: 29365321]).

Another immune hallmark of SSc is the presence of M2 macrophages (Matsushita T. et al., Expert Rev Mol Diagn. 2019 March; 19(3): 197-199. doi: 10.1080/14737159.2019.1571911. Epub 2019 Jan. 22. [PMID: 30657715]). M2 macrophages are thought to comprise a potential source of fibrosis-inducing cytokines in the skin of SSc (Higashi-Kuwata N. et al., Exp Dermatol. 2009 August; 18(8): 727-9. doi: 10.1111/j.1600-0625.2008.00828.x. Epub 2009 Mar. 3. [PMID: 19320738]), and in fact targeting M2 macrophages using T cells expressing a chimeric antigen receptor (CAR) specific for M2 surface molecules (e.g., CD206 or Fn14) in a mouse model of SSc ameliorated skin fibrosis (WO/2019/036724), suggested that M2 macrophage depletion or repolarization to M1 may help reverse immune dysfunction.

“ACVR1C” or “ALK7 is a type I receptor for the TGF beta family of signaling molecules. In humans, the TGF- family comprises at least 33 ligand genes, which are TGF-betas, activins, bone morphogenetic proteins (BMPs), and growth and differentiation factors (GDFs, nodal and lefty) (Aykul S. et al., J Biol Chem. 2016 May 13; 291(20): 10792-804. doi: 10.1074/jbc.M115.713487. Epub 2016 Mar 9. [PMID: 26961869]). The TGF-β ligands act through type I transmembrane serine/threonine kinase receptors, also termed Activin receptor-like kinases (“ALKs”; seven ALKs, ALK1 to ALK7, have been identified in mammals to date), and type II transmembrane serine/threonine kinase receptors (five type II receptors, ActRIIA, ActRIIB, BMPRII, TGFRII, and AMHRII)). Ligands can bind multiple ALKs but the affinities vary greatly: TGF-β1 binds ALK1 and ALK5 with high affinity; activin A binds ALK4 with high affinity and ALK2 and ALK7 with lower affinity; activin B mainly aims for ALK4 and ALK7; BMP4 binds ALK3 and ALK6 with high affinity and ALK2 with moderate affinity (Tengroth L. et al, Sci Rep. 2018 Jan. 24; 8(1): 1561. doi: 10.1038/s41598-018-19955-1. [PMID: 29367682]; and Morianos I et al., J Autoimmun. 2019 November; 104: 102314. doi: 10.1016/j.jaut.2019.102314. Epub 2019 Aug. 13. [PMID: 31416681]). Ligands and receptors together form a heteromeric complex that phosphorylates and activates a number of intracellular Smad transcription factors. For example, ALK7 phosphorylates Smad2/3, which phosphorylates R-Smad, which, together with Smad4, suppresses the transcription factor NFkB and the downstream transcription.

ACVR1C has been implicated in the pathology of certain diseases. For example, Bertolino et al. showed that Alk7 is a negative regulator of pancreatic beta cell function (Bertolino P. et al., Proc Natl Acad Sci USA. 2008 May 20; 105(20): 7246-51. doi: 10.1073/pnas.0801285105. Epub 2008 May 14. [PMID: 18480258]), and Li et al. showed that silencing of Alk7 alleviates cardiovascular conditions in a mouse model of type 2 diabetes (Li W. B., et al., Acta Diabetol. 2015 August; 52(4): 717-26. doi: 10.1007/s00592-014-0706-8. Epub 2015 Jan. 11. [PMID: 25577243]). Yogosawa et al. showed that Alk7 suppresses lipolysis thereby facilitating accumulation of fat in obesity through downregulation of peroxisome proliferator—activated receptor γ and C/EBPα (Yogosawa S. et al., Diabetes. 2013 January; 62(1): 115-123. [PMID: 22933117]). Michael et al. identified the Alk7 signaling as a mechanism to suppress tumorigenesis and tumor metastasis by triggering apoptosis (Michael I. P. et al., Dev Cell. 2019 May 6; 49(3): 409-424.e6. doi: 10.1016/j.devcel.2019.04.015. [PMID: 31063757]). However, to the best of the inventor's knowledge ACVRIC expression or function has not heretofore been implicated in SSc.

Therefore, based on the foregoing improved methods for treating SSc are desperately needed. If a therapy that address fibrosis and/or immune-mediated pathology of SSc is discovered, the therapy may further be useful in treating and/or preventing other fibrosis and fibrotic diseases, autoimmune diseases, and inflammatory diseases as well.

### SUMMARY OF THE INVENTION

In one aspect, the present invention in general relates to methods of treating or preventing a fibrotic, autoimmune, and/or inflammatory disease or condition in a subject.

In some embodiments, the fibrotic, autoimmune, and/or inflammatory disease or condition may be systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP). In particular embodiments, the fibrotic, autoimmune, and/or inflammatory disease or condition may be SSc.

In some embodiments, the method comprises treating a subject in need thereof with an active agent that reduces the expression or function of ACVR1C.

In some embodiments, the method comprises treating a subject in need thereof with an active agent that alters the expression or function of one or more genes listed in FIG. 2A. The one or more genes may be, NOG, ACVR1C, SOX8, GREM2, DPP4, SATB1-AS1, IL7R, DSEL, RNF157, ZFYVE9, EXOC8, TCEAL6, RAB43, ZNF217, HERPUD2, ZNF33A, PDCD4-AS1, CREBRF, PRKAG2, INADL, TCEAL2, TSPEAR-AS2, GPR155, RNF144A, SERNIC5, SATB1, EDA, LMTK3, DHRS3, ZNF658, PDE3B, TSPAN18, TLE2, AKT3, CDKN1B, DNMT3A, CEP170B, TOB1, CYTIP, DLGAP4, D2HGDH, KIF9-AS1, ARHGEF4, TTC39B, NR3C2, PLEKHB1, EPHX2, KDF1, LOC100131662, PLAG1, ZNF204P, FAM153B, PLCL1, CACNA1I, BEX5, TRPC1, MAGI3, EPHA1, ISM1, ATP6V0E2-AS1, TMEM30B, CFH, CFHR3, EXPH5, STX17-AS1, PRKCA,) XLOC_I2_010062, KIF5C, FAM134B, CMTMB, ITGA6, TRABD2A, MAL, NEFL, or TCEA3, Inc-STEAP1B-1, P2RX5, FCRL2, FFAR1, PPAPDC1B, TCF4, IL7, RRAS2, LOC100131043, PKIG, MARCH3, CXXC5, BARD1, LINC00926, TSPAN13, SLC17A9, CHST10, RHOBTB2, CYB561A3, CLCF1, SYVN1, PEAK1, DUS2, ATP5B, DEF8, FADS3, STX18, HVCN1, UCP2, PMEPA1, CD83, CRIP3, C150C15ORF57, 57, LDHA, EAF2, SNX29, BCL11A, NT5DC2, PLEKHF2, BLNK, BLK, PNOC, MS4A1, SNX22, LOC100130458, FCRLA, PCDH9, CR2, OSBPL10, CDCA7, E2F5, HS3ST1, PLEKHG1, EBF1, ABCB4, BEND4, POU2AF1, CD200, LOC101059954, NCALD, EOMES, HLA-DOB, CD19, TRAF4, CORO2B, IL6, or CD72, or any combination thereof. Optionally, at least the expression or function of ACVR1C is reduced.

In some embodiments, the method comprising reducing the expression or function of the gene product of one or more genes listed in the second half of FIG. 2A (genes shown in FIG. 2A (continued)) (NOG, ACVR1C, SOX8, GREM2, DPP4, SATB1-AS1, IL7R, DSEL, RNF157, ZFYVE9, EXOC8, TCEAL6, RAB43, ZNF217, HERPUD2, ZNF33A, PDCD4-AS1, CREBRF, PRKAG2, INADL, TCEAL2, TSPEAR-AS2, GPR155, RNF144A, SERINC5, SATB1, EDA, LMTK3, DHRS3, ZNF658, PDE3B, TSPAN18, TLE2, AKT3, CDKN1B, DNMT3A, CEP170B, TOB1, CYTIP, DLGAP4, D2HGDH, KIF9-AS1, ARHGEF4, TTC39B, NR3C2, PLEKHB1, EPHX2, KDF1, LOC100131662, PLAG1, ZNF204P, FAM153B, PLCL1, CACNA1I, BEX5, TRPC1, MAGI3, EPHA1, ISM1, ATP6V0E2-AS1, TMEM30B, CFH, CFHR3, EXPH5, STX17-AS1, PRKCA, XLOC_I2_010062, KIF5C, FAM134B, CMTMB, ITGA6, TRABD2A, MAL, NEFL, or TCEA3, or any combination thereof). Optionally, the expression or function of the gene product of ACVR1C, NR3C2, LOC100131662 and/or CFHR3 may be reduced. Optionally, the expression or function of the gene product of ACVR1C, GREM2, NOG, and/or ZFYVE9 may be reduced. Optionally, the expression or function of the gene product of ACVR1C and/or optionally IL7R and/or DNMT3A may be reduced.

In some embodiments, the method comprises increasing or enhancing the expression or function of the gene product of one or more genes listed in the first half of FIG. 2A (Inc-STEAP1B-1, P2RX5, FCRL2, FFAR1, PPAPDC1B, TCF4, IL7, RRAS2, LOC100131043, PKIG, MARCH3, CXXC5, BARD1, LINC00926, TSPAN13, SLC17A9, CHST10, RHOBTB2, CYB561A3, CLCF1, SYVN1, PEAK1, DUS2, ATP5B, DEF8, FADS3, STX18, HVCN1, UCP2, PMEPA1, CD83, CRIP3, C150C15ORF57, 57, LDHA, EAF2, SNX29, BCL11A, NT5DC2, PLEKHF2, BLNK, BLK, PNOC, MS4A1, SNX22, LOC100130458, FCRLA, PCDH9, CR2, OSBPL10, CDCA7, E2F5, HS3ST1, PLEKHG1, EBF1, ABCB4, BEND4, POU2AF1, CD200, LOC101059954, NCALD, EOMES, HLA-DOB, CD19, TRAF4, CORO2B, IL6, CD72, or any combination thereof). Optionally, the expression or function of the gene product of E2F5, CD19, IL6, ZNF204P, PLEKHF2, or any combination thereof.

In some embodiments, the method comprises administering hematopoietic stem cell transplantation (HSCT) to the subject. Optionally HSCT reduces or increases or further reduces or increases the expression or function of said one or more genes the expression or function of which is to be reduced or increased by an active agent. Further optionally, HSCT modifies or further modifies the expression or function of one or more genes of the TGF-beta signaling pathway in the treated subject.

In some embodiments, the method comprises altering the expression or function of the gene product of one or more genes of the TGF-beta signaling pathway. In certain embodiments, the altering comprises one or more of the following: (i) decreasing the expression or function of ACVR1C; (ii) decreasing the expression or function of GREM2; (iii) decreasing the expression or function of NOG; (iv) decreasing the expression or function of ZFYVE9; and/or (v) increasing or blocking the expression or function of E2F5.

In some embodiments, the method comprises one or more of the following: (i) decreasing the expression or function of ACVR1C; (ii) decreasing the expression or function of GREM2; (iii) decreasing the expression or function of NOG; (iv) decreasing the expression or function of ZFYVE9; (v) increasing the expression or function of E2F5; (vi) decreasing the expression or function of NR3C2; (vii) decreasing the expression or function of LOC100131662; (viii) decreasing the expression or function of CFHR3; (ix) decreasing the expression or function of IL7R; (x) increasing the expression or function of CD19; (xi) increasing the expression or function of IL6; (xii) decreasing the expression or function of DNMT3A; (xiii) increasing the expression or function of ZNF204P; and/or (xiv) increasing the expression or function of PLEKHF2.

In some embodiments, the method comprises administering to the subject: (a) an agent that decreases the expression or function of ACVR1C, GREM2, NOG, ZFYVE9, NR3C2, LOC100131662, CFHR3, IL7R, and/or DNMT3A; and/or (b) an agent that increases the expression or function of E2F5, CD19, IL6, ZNF204P, and/or PLEKHF2.

In some embodiments, the method comprises administering to the subject a composition comprising (a) an agent that decreases the expression or function of ACVR1C, GREM2, NOG, ZFYVE9, NR3C2, LOC100131662, CFHR3, IL7R, and/or DNMT3A; and/or (b) an agent that increases the expression or function of E2F5, CD19, IL6, ZNF204P, and/or PLEKHF2.

In the methods of treating or preventing SSc according to the present invention, the agent administered to reduce or increase gene expression or function may be an antibody, an antigen-binding antibody fragment (e.g., scFv, Fab, Fab′, (Fab′)₂), a chimeric antigen receptor (CAR)-expressing cell, an siRNA, an shRNA, an miRNA, an aptamer, a CRISPR/Cas-based gene therapy agent, a peptide, a small molecule, a polymer, an expression vector encoding a gene of interest, or any combination thereof. In some embodiments, the agent may be a blocking or antagonistic antibody or an antigen-binding antibody fragment (e.g., scFv, Fab, Fab′, (Fab′)₂) specific for ACVR1C, GREM2, NOG, ZFYVE9, NR3C2, LOC100131662, CFHR3, IL7R, and/or DNMT3A. In certain embodiments, the agent may be a blocking or antagonistic antibody or an antigen-binding antibody fragment (e.g., scFv, Fab, Fab′, (Fab′)₂) specific for ACVR1C.

In some embodiments, the method further comprises administering at least one other active agent. Optionally, the other active agent is an anti-inflammatory agent, an immunosuppressant, an anti-fibrotic agent, a vasodilator, and/or an analgesic. In certain embodiments, the at least one other active agent may be nintedanib, an NSAID, a corticosteroid, methotrexate, cyclosporine, anti-thymocyte globulin, mycophenolate mofetil and cyclophosphamide, a calcium channel blocker (e.g., nifedipine), an angiotensin converting enzyme inhibitor (ACE inhibitor), an endothelin-1 receptor inhibitor (e.g., bosentan), a prostaglandin (e.g., epoprostenol, prostacyclin), nitric oxide, or a collagen inhibitor (e.g., colchicine, para-aminobenzoic acid (PABA), dimethyl sulfoxide, and D-penicillamine), or any combination thereof.

In some embodiments, the method further comprises (I) increasing or decreasing memory B cells, naïve B cells, and/or CD8+ T cells; and/or (II) increasing or decreasing memory CD4+ T cells, resting CD4+ T cells, and/or naïve CD4+ T cells; and/or (III) increasing or decreasing innate immune cells. Optionally, in (III), the innate immune cells may be monocytes, macrophages, and/or dendritic cells. In certain embodiments, the (I) and/or (II) may be achieved by the active agent that alters the expression or function of the gene product of any one of the genes listed in FIG. 2A or by the at least one active agent, or by the combination of the active agent and the at least one other active agent. Alternatively, another active agent that provides (I) and/or (II) may be administered to the subject.

In further embodiments, the method further comprises detecting the expression or function of one or more of the genes the expression of which is to be increased or decreased, wherein said detecting occurs prior, during and/or after treatment. In yet further embodiments, the method further comprises detecting changes in immune cells which is to be increased (memory B cells, naïve B cells, and/or CD8+ T cells) or decreased (memory CD4+ T cells, resting CD4+ T cells, and/or naïve CD4+ T cells), wherein said detecting occurs prior, during and/or after treatment.

The expression or function of the gene product of one or more of the genes and/or immune cell numbers and/or percentages may be detected in one or more samples from the treated subject, optionally a blood sample, a skin sample, a vascular sample, a gastrointestinal tract sample, a lung sample, a heart sample, and/or a kidney sample. In some instances, the gene expression may be detected and evaluated using methods as described in Example 1. For a large set of genes, DNA microarrays may be used. For a small number of genes, a standard q-PCR method may be used. In some instances, immune cell quantification may be done using a standard flow cytometry method. Alternatively, or additionally, immune cells may be quantified via cell and/or tissue histology using cytospun samples and/or tissue slices.

In another aspect, the present invention relates to methods of determining whether a therapy for a fibrotic, autoimmune, and/or inflammatory disease or condition is effective in a subject. The fibrotic, autoimmune, and/or inflammatory disease or condition may be any one or more of the diseases and conditions described above. In some embodiments, the method comprises (a) measuring the expression of one or more genes listed in FIG. 2A in a sample from the subject before and at one or more time points after starting the therapy and (b) determining that the therapy is effective if: (i) one or more genes listed in the second half of FIG. 2A (genes shown in FIG. 2A (continued)) are downregulated at least one time point after starting the therapy compared to before starting the therapy; and/or (ii) one or more genes listed in the first half of FIG. 2A are upregulated at least one time point after starting the therapy compared to before starting the therapy. In some embodiments, the sample used the method may be a blood sample, a skin sample, a vascular sample, a gastrointestinal tract sample, a lung sample, a heart sample, and/or a kidney sample.

In certain embodiments, the immune cell quantity in the sample may be another criterion for evaluating therapy effectiveness. In such an embodiment, the method may comprise: (a) measuring the expression of one or more genes listed in FIG. 2A in a sample from the subject before and at one or more time points after starting the therapy; (b) quantifying the numbers and/or percentage of memory B cells, naïve B cells, memory CD4+ T cells, resting CD4+ T cells, naïve CD4+ T cells, and/or CD8+ T cells in a sample from the subject before and at one or more time points after starting the therapy. The method may further comprise (c) determining that the therapy is effective if: (i) one or more genes listed in the second half of FIG. 2A (genes shown in FIG. 2A (continued)) are downregulated at least one time point after starting the therapy compared to before starting the therapy; and/or (ii) one or more genes listed in the first half of FIG. 2A are upregulated at least one time point after starting the therapy compared to before starting the therapy, and if: (I) the number and/or percentage of memory B cells, naïve B cells, and/or CD8+ T cells is increased relative to a healthy control; and/or (II) the number and/or percentage of memory CD4+ T cells, resting CD4+ T cells, and/or naïve CD4+ T cells, is decreased relative to a healthy control. In some embodiments, the sample in (a) and/or (b) may be a blood sample, a skin sample, a vascular sample, a gastrointestinal tract sample, a lung sample, a heart sample, and/or a kidney sample.

In certain embodiments, the one or more genes in (i) may be ACVR1C, GREM2, NOG, ZFYVE9, NR3C2, LOC100131662, CFHR3, IL7R, and/or DNMT3A, and/or the one or more genes in (ii) may be E2F5, CD19, IL6, ZNF204P, and/or PLEKHF2.

In another aspect, the present invention relates to methods of screening for a therapeutic agent for a fibrotic, autoimmune, and/or inflammatory disease or condition. The fibrotic, autoimmune, and/or inflammatory disease or condition may be any one or more of the diseases and conditions described above. In some embodiments, the method comprises: (a) applying a candidate therapeutic agent to (I) one or more cells derived from a patient having the fibrotic, autoimmune, and/or inflammatory disease or condition, (II) one or more cell line cells of (or representing) the fibrotic, autoimmune, and/or inflammatory disease or condition, or (III) a cell or tissue culture comprising a sample derived from a patient having the fibrotic, autoimmune, and/or inflammatory disease or condition; and (b) after step (a), measuring the expression of one or more genes listed in FIG. 2A in (I) the one or more cells derived from a patient, (II) the one or more cell line cells, or (III) the cell or tissue culture comprising a sample derived from a patient. The method may further comprise (c) determining that a candidate therapeutic agent is effective if: (i) one or more genes listed in the second half of FIG. 2A (genes shown in second page of FIG. 2A (continued)) are downregulated compared to an untreated or placebo control; and/or (ii) one or more genes listed in the first page of FIG. 2A are upregulated compared to an untreated or placebo control.

In certain embodiments, the one or more genes in (i) may be ACVR1C, GREM2, NOG, ZFYVE9, NR3C2, LOC100131662, CFHR3, IL7R, and/or DNMT3A, and/or the one or more genes in (ii) may be E2F5, CD19, IL6, ZNF204P, and/or PLEKHF2.

In certain embodiments, the one or more cells in (I) or (II) may comprise a skin cell (e.g., a keratinocyte), a fibroblast, a blood cell, an immune cell, a macrophage, a vascular ell, a gastrointestinal cell, a lung cell, a heart cell, and/or a renal cell. In certain embodiments, the cell or tissue culture in (III) comprises a skin tissue, an organoid, or a three-dimensional layered cell culture. In certain embodiments, the sample derived from a patient in (III) comprises: (i) one or more fibroblasts derived from a patient; (ii) one or more macrophages derived from a patient; and/or (iii) serum or plasma derived from a patient. In further embodiments, the cell or tissue culture in (III) is a three-dimensional, skin-like layered cell culture comprising: (i) one or more fibroblasts; and (ii) one or more keratinocytes; and optionally (iii) one or more monocytes or macrophages; and/or (iv) serum or plasma, wherein at least one of (i)-(iv) is derived from a patient. Optionally, the cell or tissue culture in (III) is maintained in a transwell plate. Further optionally, the cell or tissue culture in (III) is a self-assembling three-dimensional culture, which optionally mimics affected skin in SSc.

In yet another aspect, the present invention relates to methods of predicting whether a subject with a fibrotic, autoimmune, and/or inflammatory disease or condition will respond to HSCT. The fibrotic, autoimmune, and/or inflammatory disease or condition may be any one or more of the diseases and conditions described above. In certain embodiments, the disease and/or condition is SSc. In some embodiments, the method comprises: (a) measuring the expression of ACVR1C in a sample from the subject; and (b) determining that the subject will be a good responder to HSCT if the subject is a high expresser of ACVR1C. Optionally, the SSc the subject has is fibroproliferative, normal-like SSc, or inflammatory SSc. The subset definition is according to Milano A. et al., (Milano A. et al., PLoS One. 2008 Jul. 16; 3(7): e2696. doi: 10.1371/journal.pone.0002696. [PMID: 18648520], which refers to fibroproliferative as diffuse-proliferation or diffuse-proliferative).

In yet another aspect, the present invention relates to methods of treating a subject having a fibrotic, autoimmune, and/or inflammatory disease or condition. The fibrotic, autoimmune, and/or inflammatory disease or condition may be any one or more of the diseases and conditions described above. In certain embodiments, the disease and/or condition is SSc. The method may comprise: (a) measuring the expression of ACVR1C in a sample from the subject; and (b) treating the subject with hematopoietic stem cell transplant (HSCT) if the subject is a high expresser of ACVR1C. Optionally, the subject has fibroproliferative, normal-like SSc, or inflammatory SSc.

In yet another aspect, the present invention relates to methods of predicting whether a subject with a fibrotic, autoimmune, and/or inflammatory disease or condition will respond to cyclophosphamide (CYC) treatment. The fibrotic, autoimmune, and/or inflammatory disease or condition may be any one or more of the diseases and conditions described above. In certain embodiments, the disease and/or condition is SSc. In some embodiments, the method comprises: (a) measuring the expression of CLCF1 in a sample from the subject; and (b) determining that the subject will be a good responder to CYC if the subject is a high expresser of CLCF1.

In yet another aspect, the present invention relates to methods of treating a subject having a fibrotic, autoimmune, and/or inflammatory disease or condition. The fibrotic, autoimmune, and/or inflammatory disease or condition may be any one or more of the diseases and conditions described above. In certain embodiments, the disease and/or condition is SSc. In some embodiments, the method comprises: (a) measuring the expression of CLCF1 in a sample from the subject; and (b) treating the subject with CYC if the subject is a high expresser of CLCF1.

Optionally, in any of the methods of predicting and/or methods of treating, the sample is a blood sample, a skin sample, a vascular sample, a gastrointestinal tract sample, a lung sample, a heart sample, and/or a kidney sample.

In a further aspect, the present invention relates to agents for treating or preventing a fibrotic, autoimmune, and/or inflammatory disease or condition. The fibrotic, autoimmune, and/or inflammatory disease or condition may be any one or more of the diseases and conditions described above. In certain embodiments, the disease and/or condition is SSc. Such an agent may be (i) one which decreases the expression of one or more genes listed in the second half of FIG. 2A (genes shown in FIG. 2A (continued)); (ii) one which suppresses, blocks, or inhibits the function of the gene product of one or more genes listed in the second half of FIG. 2A (genes shown in FIG. 2A (continued)); (iii) one which decreases the expression or function of ACVR1C, GREM2, NOG, ZFYVE9, NR3C2, LOC100131662, CFHR3, IL7R, and/or DNMT3A; (iv) one which increases the expression of one or more genes listed in the first half of FIG. 2A; (v) one which enhances the function of the gene product of one or more genes listed in the second half of FIG. 2A (genes shown in FIG. 2A (continued)); (vi) one which enhances the expression or function of E2F5, CD19, IL6, ZNF204P, and/or PLEKHF2; or any combination of the foregoing. In some embodiments, the agent optionally is selected from an antibody, an antigen-binding antibody fragment (e.g., scFv, Fab, Fab′, (Fab′)₂), a chimeric antigen receptor (CAR)-expressing cell, an siRNA, an shRNA, an miRNA, an aptamer, a CRISPR/Cas-based gene therapy agent, a peptide, a small molecule, a polymer, an expression vector encoding a gene of interest, or any combination thereof.

In certain embodiments, the agent comprises (i) a neutralizing, blocking, and/or antagonistic antibody or antigen-binding antibody fragment specific for ACVR1C, GREM2, NOG, ZFYVE9, NR3C2, LOC100131662, CFHR3, IL7R, and/or DNMT3A, (ii) a neutralizing, blocking, and/or antagonistic antibody or antigen-binding antibody fragment specific for ACVR1C, which optionally is designed to specifically or preferentially neutralize, block, and/or antagonize ACVR1C on macrophages, fibroblasts, and/or keratinocytes; (iii) an agonistic antibody or antigen-binding antibody fragment specific for E2F5, CD19, IL6, ZNF204P, and/or PLEKHF2 or (iv) any combination of any of the foregoing, or a composition containing any of the foregoing.

In a yet further aspect, the present invention relates to kits. In some embodiments, the kit may comprise (a) at least one primer set for detecting expression of at least one gene listed in FIG. 2A; and (b) an instruction sheet. Such a kit may be used, for example, for determining the expression of one or more particular genes listed in FIG. 2A (e.g. ACVR1C) or the gene expression signature in a sample, for example a sample derived from a subject with a fibrotic, autoimmune, and/or inflammatory disease or condition or a cell line of (or representing) a fibrotic, autoimmune, and/or inflammatory disease or condition. The fibrotic, autoimmune, and/or inflammatory disease or condition may be any one or more of the diseases and conditions described above. In certain embodiments, the disease and/or condition is SSc. The kit may be useful in determining the treatment effectiveness in a subject, predicting whether a subject will respond to a certain therapy for a fibrotic, autoimmune, and/or inflammatory disease or condition such as but not limited to SSc (e.g. HSCT), or evaluating whether a candidate therapeutic agent would be effective in treating or preventing a fibrotic, autoimmune, and/or inflammatory disease or condition such as but not limited to SSc.

In some embodiments, the kit may comprise: (a) (I) one or more cells derived from a patient having a fibrotic, autoimmune, and/or inflammatory disease or condition, (II) one or more cell line cells having a fibrotic, autoimmune, and/or inflammatory disease or condition, or (III) a cell or tissue culture comprising a sample derived from a patient having a fibrotic, autoimmune, and/or inflammatory disease or condition; and (b) at least one primer set for detecting expression of at least one gene listed in FIG. 2A. Optionally, the kit may be for screening a therapeutic agent for treating a fibrotic, autoimmune, and/or inflammatory disease or condition such as SSc.

In any of the embodiments related to a kit, the at least one gene optionally comprises ACVR1C.

### BRIEF DESCRIPTION OF THE DRAWINGS

FIGS. 1A-1B: Limitation of missing data in the SCOT trial. FIG. 1A provides a bar graph showing the numbers of patients with gene expression data at each time point. Dark green, blue and red stands for healthy control, transplant and cyclophosphamide, respectively. FIG. 1B provides a Venn diagram showing the overlapped differential expressed genes between three analyses using different variants of these data: 1) using the patients with only complete data (referred to as Complete), 2) using the original data with missing time points (refer as Original) and 3) using the imputed data (refer as Impute).

FIGS. 2A-2J: Examples of differential expressed genes and pathway between two treatments. FIG. 2A provides a heatmap of all 142 differential expressed genes using gene expression data from peripheral blood cells (PBCs) (yellow is high expression and blue is low expression). Blue and red bars on the top stand for transplant and cyclophosphamide, respectively. Filled arrows indicate genes that were differentially expressed in all three different data sets (Complete, Original, and Impute). Open arrows indicate genes that belong to the TGF beta signaling pathway. Dotted arrows indicate genes whose expression changes are shown in FIGS. 2B-2E. FIGS. 2B-2E provide exemplary gene expression changes over time for ACVR1C, IL7R, CD19, and IL6., respectively. FIG. 2F compares TGF-beta signaling pathway expression between two treatments along with time points using average expression of those five genes. Wilcoxon=test p values were listed. FIGS. 2G-2I provide exemplary gene expression changes over time for other four TGF-beta signaling pathway genes, GREM2, ZFYVE9, NOG, and E2F5, respectively. Blue and red boxes/lines stand for transplant and cyclophosphamide, respectively. The shaded region corresponds to the standard error of the mean at each timepoint.

FIGS. 3A-3E: Longitudinal analysis of treatment effects on relative cell type proportions. Mixed effect linear models were used to identify cell types that were significantly associated with treatment. The cell type proportion for memory B cells (FIG. 3A), naïve B cell (FIG. 3B), resting memory CD4 T cell (FIG. 3C), naïve CD4 T cell (FIG. 3D), and CD8 T cell (FIG. 3E) are shown over time by the solid lines (HSCT in blue, CYC in red). The shaded region corresponds to the standard error of the mean at each timepoint.

FIGS. 4A-4D: Survival plots using SCOT baseline patients. Patients were divided into high and low expression groups based on the median expression of a gene. Log-rank test p values and hazard ratios (HR) were listed. FIGS. 4A-4B are plots for ACVR1 C in transplant and cyclophosphamide FIGS. 4C-4D are plots for CLCF1 in transplant and cyclophosphamide.

FIGS. 5A-5D: Comparisons of ACVR1C expression changes caused by transplant in autoimmune diseases. FIG. 5A provides ACVR1C expression changes in SSc patients in the SCOT trial, and FIG. 5B provides ACVR1C expression changes in Crohn's disease (CD) patients. FIG. 5C provides ACVR1C expression changes in CD4+ T cells in Multiple Sclerosis (MS) patients, and FIG. 5D provides ACVR1C expression changes in CD8+ T cells in MS patients. p values were determined by the Wilcoxon test. All time points were collected from the original datasets. To compare the fold-change of ACVR1C, y-axes were plotted on the same scale.

FIGS. 6A-6C: ACVR1C expression changes and co-expression networks in ‘intrinsic’ subsets. ACVR1C expression changes in the SCOT trial in patients of the proliferative (FIG. 6A), normal-like (FIG. 6B), and Inflammatory (FIG. 6C) subsets are provided. Blue and red lines stand for transplant and cyclophosphamide, respectively. Also provided are co-expression networks for proliferative (FIG. 6D), normal-like (FIG. 6E), and inflammatory (FIG. 6F) subsets. The clusters of TGF-beta genes were highlighted. Correlations were calculated between the five TGF-beta genes and the rest genes. Only genes with correlation greater or lesser than 0.3 and p values <0.01 were considered.

FIGS. 7A-7C provide the gene expression trends of differential expressed genes between two treatments (cyclophosphamide (red) and hematopoietic stem cell transplant (blue)). Differentially expressed genes were identified by using linear mixed regression algorithm between two treatments using three different variants of the data: 1) using the patients with only complete data (referred to as Complete) (FIG. 7A), 2) using the original data with missing time points (refer as Original) (FIG. 7B), and 3) using the imputed data (refer as Impute) (FIG. 7C). These four genes are shared by the three comparisons. All plots are significant (all FDR<0.05).

FIGS. 8A-8D provide boxplots for pathway expression comparisons between two treatments (cyclophosphamide (red) and hematopoietic stem cell transplant (blue)) over time. The analyzed pathways were the hematopoietic cell lineage pathway (FIG. 8A), B cell receptor signaling pathway (FIG. 8B), Epstein-Barr virus infection pathway (FIG. 8C), and HIF-1 signaling pathway (FIG. 8D). P values were listed for only significant comparisons (p<0.05).

FIGS. 9A-9C provide graphs that demonstrate the gene expression of DNMT3A, ZNF204P, and PLEKHF2 predicts survival outcomes in SSc. Survival plots for DNMT3A (FIG. 9A), ZNF204P (FIG. 9B), and PLEKHF2 (FIG. 9C). Patients were divided into high (green) and low (red) expression groups based on the median expression of a gene and event free survival (EFS) was tracked. Log-rank test p values and hazard ratios (HR) were listed.

FIGS. 10A-10B provide ACVR1C expression comparisons in Crohn's disease patients who received HSCT. The data were collected from GSE100922. FIG. 10A shows expression changes in HSCT responders and FIG. 10B shows expression changes in HSCT non-responders. Responders and non-responders were defined in the original paper (Corraliza A. M. et al., J Crohns Colitis. 2019 Apr. 26; 13(5):634-647. doi: 10.1093/ecco-jcc/jjy203. [PMID:30521002]). P values were calculated by the Wilcoxon-test.

FIG. 11 provides ACVR1C expression comparisons of ‘intrinsic’ subsets in SCOT. P values were calculated by the Wilcoxon-test.

### DETAILED DESCRIPTION OF THE INVENTION

The Scleroderma: Cyclophosphamide or Transplantation (SCOT) trial demonstrated that patients who underwent hematopoietic stem cell transplantation (HSCT) had better outcomes than those treated with cyclophosphamide (CYC) (Sullivan K. M. et al., N Engl J Med. 2018 Jan. 4; 378(1): 35-47. doi: 10.1056/nejmoa1703327. [PMID: 29298160]). Applicant subsequently demonstrated that patients who had the most significant difference in event free survival (EFS) were patients assigned to the fibroproliferative intrinsic subset using PBC samples (Franks J. M. et al., Arm Rheum Dis. 2020 Sep. 15; annrheumdis-2020-217033. doi: 10.1136/annrheumdis-2020-217033. Online ahead of print. [PMID: 32933919]).

In the present disclosure, Applicant systematically examined gene expression changes in the SCOT trial participants over time between the two treatment arms, CYC and HSCT. Differentially expressed genes were identified between treatment arms using linear mixed regression model and, for example, significant enrichment for TGFb signaling downregulation in the HSCT arm was observed. Based on the discovery, Applicant discloses herein therapeutic and/or prophylactic methods, diagnosis methods, therapeutic effect evaluation methods, therapeutic agent screening methods, therapy response prediction methods, therapy selection methods based on gene expression, therapeutic and/or prophylactic agents, compositions, and kits, which are useful for treating, preventing, evaluating therapeutic effects for, identifying effecting therapeutic agents for, predicting therapy responses in, selecting an appropriate therapy for a patient with SSc and also potentially other diseases that cause a similar condition and/or symptom to that of SSc.

### Therapeutic and/or Prophylactic Methods

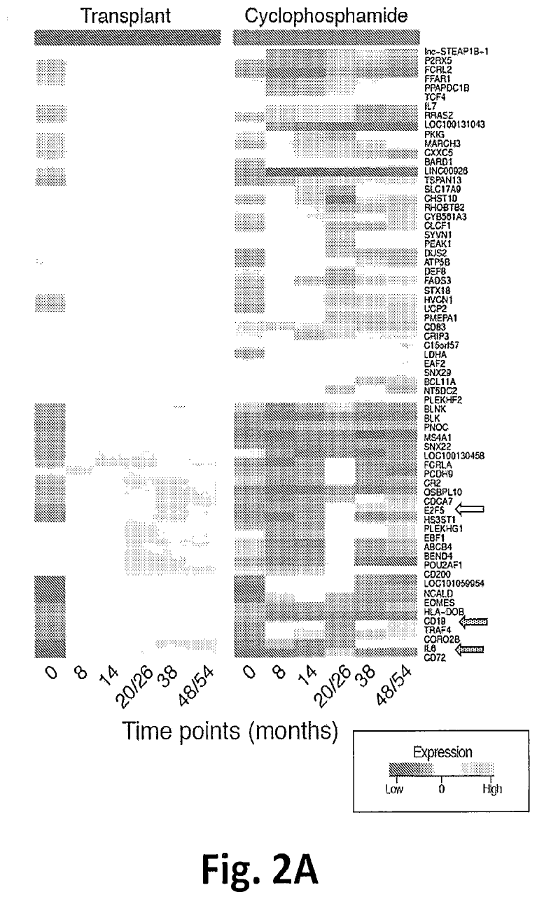

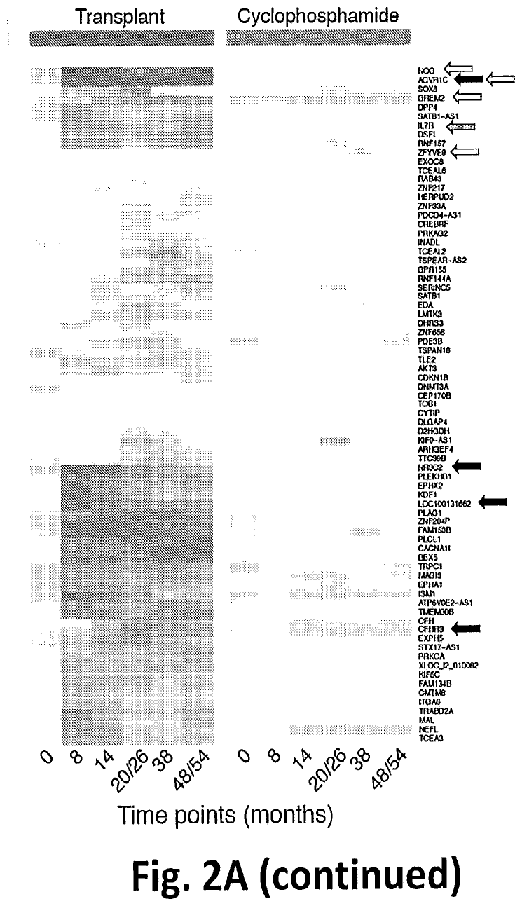

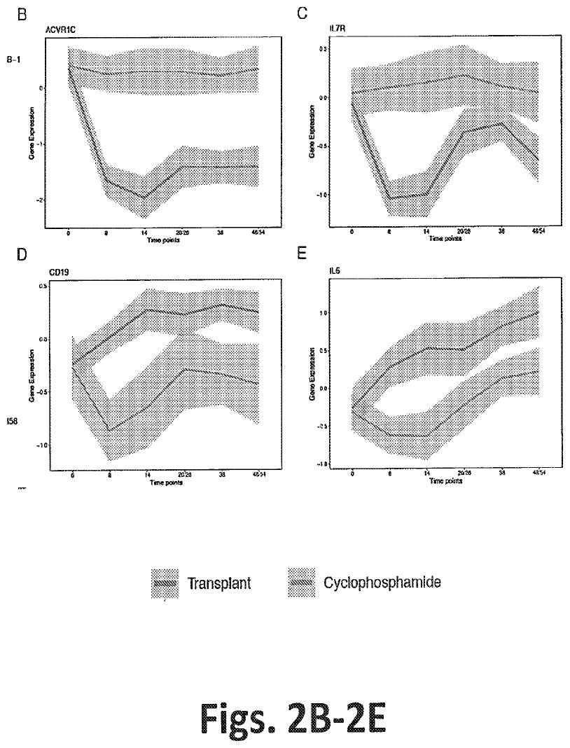

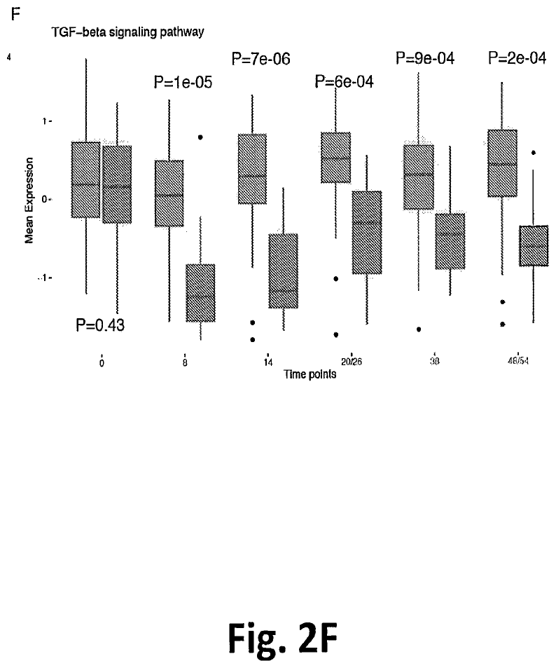

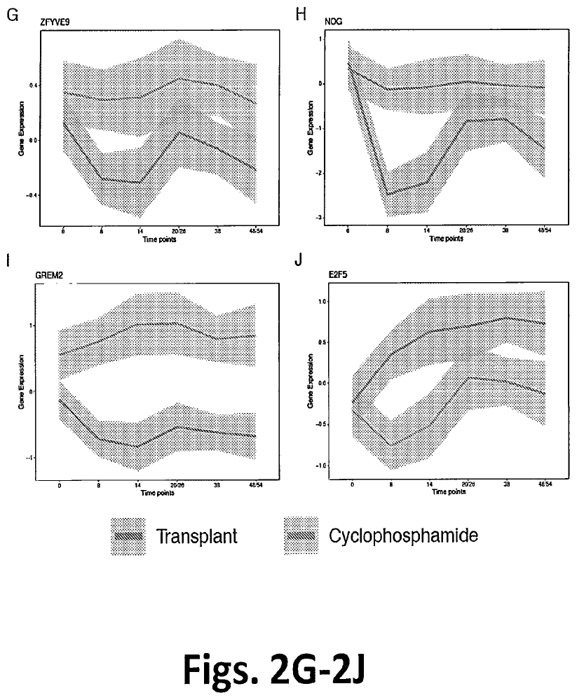

An aspect of the invention relates to methods of treating or preventing SSc in a subject. Applicant discovered that expression of genes listed in FIG. 2A are altered in SSc during the HSCT or CYC treatment, using samples form a clinical trial that showed HSCT treatment outperforms CYC treatment. Applicant envisions that modifying the expression or function of the gene product of genes that were altered in the HSCT group provide an alternative therapeutic and/or prophylactic method that may be less invasive, less costly, and/or less risky compared to HSCT. Alternatively, such modifying may further enhance the efficacy of HSCT. Further alternatively, HSCT may be used to alter gene expression. In some embodiments, expression of at least one of the genes listed in FIG. 2A may be altered by administering an active agent, or the function of the gene product of at least one of the genes listed in FIG. 2A may be altered by administering an active agent.

Applicant discovered herein that the genes listed in the second half of FIG. 2A (genes in FIG. 2A (continued)) were downregulated in SSc patients who received HSCT than SSc patients who received CYC, where HSCT provided better a therapeutic outcome than CYC. Therefore, according to some embodiments, SSc may be treated or prevented by administering a subject in need thereof an agent that reduces the expression of or inhibiting the function of the genes listed in the second half of FIG. 2A (genes in second page FIG. 2A (continued)).

Among three different data sets (“complete”, “original” and “impute”; see Example 1) comparing HSCT vs CYC, reduction in expression of ACVR1C, NR3C2, LOC100131662 and CFHR3 in HSCT was commonly observed. Based on this, administering an agent that reduces the expression (and/or function of the gene product) of ACVR1C, NR3C2, LOC100131662 and/or CFHR3, or any combination thereof may treat or prevent SSc in a subject.

According to the gene enrichment analyses, genes associated with the TGF beta signaling pathway showed the greatest and most consistent reduction in expression in SSc patients who received HSCT. Among the genes listed in the second half of FIG. 2A (genes in FIG. 2A (continued)), the TGF beta signaling pathway genes are ACVR1C, GREM2, NOG, and ZFYVE9. Accordingly, in some embodiments, administering an agent that reduces the expression (and/or function of the gene product) of ACVR1C, GREM2, NOG, and/or ZFYVE9 may treat or prevent SSc in a subject.

Particularly, expression and/or function of ACVR1C may be reduced by administering an agent to treat or prevent SSc in a subject. In certain embodiments, interaction of ACVR1C with its ligand(s) may be inhibited or the ligand(s) may be targeted. Such a ligand may be activin A or activin B or BMP7. Alternatively, downstream molecules of ACVR1C may be inhibited. For example, the downstream molecule may be Smad2, Smad3, R-Smad, or Smad 4.

IL7R and DNMT3A were also among the genes that had low expression in HSCT and high expression in CYC. In some embodiments, administering an agent hat reduce the expression (and/or function of the gene product) of IL7R and/or DNMT3A may treat or prevent SSc in a subject.

In some embodiments, expression (and/or function of the gene product) of ACVR1C and at least one more gene may be reduced by administering an agent. The at least one more gene may be NR3C2, LOC100131662, CFHR3, GREM2, NOG, ZFYVE9, IL7R, or DNMT3A, or any combination thereof.

Reduction in the gene expression (e.g., ACVR1C gene expression) may be about 25% reduction, about 30% reduction, about 35% reduction, about 40% reduction, about 45% reduction, about 50% reduction, about 55% reduction, about 60% reduction, about 65% reduction, about 70% reduction, about 75% reduction, about 80% reduction, about 85% reduction, about 90% reduction, about 95% reduction, or about 100% reduction.

In some embodiments, the treatment and/or prophylactic methods may comprise administering the subject an agent that reduces the expression or function of the one or more genes listed in the second half of FIG. 2A (genes in FIG. 2A (continued)) or administering the subject a composition comprising such an agent. In some embodiments, the subject may receive HSCT.

Applicant also discovered herein that the genes listed in the first half of FIG. 2A were upregulated in SSc patients who received HSCT than SSc patients who received CYC, where HSCT provided a better therapeutic outcome than CYC. Therefore, according to some embodiments, administering an agent that increases the expression or enhancing the function of the gene product of the genes listed in the first half of FIG. 2A may treat or prevent SSc in a subject.

E2F5, was among the upregulated genes in SSc patients who received HSCT. Therefore, in some embodiments, administering an agent that increases the expression or enhancing the function of the gene product of E2F5 may treat or prevent SSc in a subject. E2F5 (ELL protein-associated factor 2) is a gene of the TGF beta signaling pathway, and E2F5 was previously reported to inhibit TGF beta signaling through a direct interaction with Smad 3 (Liu X. et al. J Biol Chem. 2015 Oct. 23; 290(43): 25933-25945. Published online 2015 Sep. 14. doi: 10.1074/jbc.M115.663542 [PMID: 26370086]). Positive association discovered by Applicant herein between E2F5 expression and better therapeutic outcome is surprising because TGF beta has been implicated as a pathological and/or pathogenic factor in SSc and anti-TGF beta monoclonal antibody therapy (fresolimumab) in fact decreased SSc biomarkers and improved clinical symptoms in SSc patients (Rice L. M. et al., J Clin Invest. 2015 Jul. 1; 125(7): 2795-2807. Published online 2015 Jun. 22. doi: 10.1172/JCI77958 [PMID: 26098215]). Without wishing to be bound by a theory, it is possible that some member(s) of the TGF beta signaling pathway contribute to the pathology of SSc and some member(s) of the TGF beta signaling pathway function to negate the pathology of SSc, especially considering the complexity of the TGF beta signaling pathway.

Expression of CD19, IL6, ZNF204P, and PLEKHF2 was also upregulated in SSc patients who received HSCT. Therefore, in some embodiments, administering an agent that increases the expression or enhancing the function of the gene product of CD19, IL6, ZNF204P, and PLEKHF2 may treat or prevent SSc in a subject.

According to the inventive methods, the increase in the gene expression (e.g., ACVR1C gene expression) may be about 25% increase, about 30% increase, about 35% increase, about 40% increase, about 45% increase, about 50% increase, about 55% increase, about 60% increase, about 65% increase, about 70% increase, about 75% increase, about 80% increase, about 85% increase, about 90% increase, about 95% increase, about 100% increase, about 150% increase, about 200% increase, about 300% increase, about 400% increase, about 500% increase, about 600% increase, about 700% increase, about 800% increase, about 900% increase, about 1000% increase, about 2000% increase, about 3000% increase, about 4000% increase, or about 5000% increase.

In some embodiments, the treatment and/or prophylactic methods may comprise administering the subject an agent that enhances the expression or function of the one or more genes listed in the first half of FIG. 2A or administering the subject a composition comprising such an agent. In some embodiments, the subject may receive HSCT.

Applicant's gene enrichment analyses described herein in Example 1 revealed that genes associated with the TGF beta signaling pathway had the greatest and most consistent reduction in expression in SSc patients who received HSCT. Specifically, ACVR1C, GREM2, NOG, and ZFYVE9 were downregulated and E2F5 was upregulated. Therefore, in some embodiments, the treatment and/or prophylactic methods according to the present invention may comprise one or more of: (i) decreasing the expression or function of ACVR1C; (ii) decreasing the expression or function of GREM2; (iii) decreasing the expression or function of NOG; (iv) decreasing the expression or function of ZFYVE9; and/or (v) increasing the expression or function of E2F5, which may be achieved by administering an agent that provide any one or more of (i)-(v). As described above, TGF beta has been implicated as a pathological and/or pathogenic factor in SSc and anti-TGF beta monoclonal antibody therapy (fresolimumab) in fact decreased biomarkers and improved clinical symptoms in SSc patients (Rice L. M. et al., J Clin Invest. 2015 Jul. 1; 125(7): 2795-2807. Published online 2015 Jun. 22. doi: 10.1172/JCI77958 [PMID: 26098215]). Therefore, in some embodiments, the treatment and/or prophylactic methods according to the present invention may further comprise administering an anti-TGF beta Ab. Such a combination of an anti-TGF beta Ab therapy and modification of TGF beta signaling pathway genes (e.g., modifications as described in any one or more of (i)-(v)) may further attenuate the pathological mechanisms caused by the TGF beta family members and their downstream signaling effects. In certain embodiments, modification of the TGF beta signaling pathway genes may be achieved by HSCT.

In some embodiments, any one or more of ACVR1C, GREM2, NOG, ZFYVE9, NR3C2, LOC100131662, CFHR3, IL7R, and/or DNMT3A may be reduced in its expression and/or function, and/or any one or more of E2F5, CD19, IL6, ZNF204P, and/or PLEKHF2 may be increased in its expression and/or function. This may be achieved by administering to the subject an agent that causes such a change(s) or a composition comprising such an agent.

In some embodiments, reducing or increasing the expression and/or function of the gene product of any one of the genes listed in FIG. 2A may be via administering an agent. The agent may be an antibody, an antigen-binding antibody fragment (e.g., scFv, Fab, Fab', (Fab')2), a chimeric antigen receptor (CAR)-expressing cell, an siRNA, an shRNA, an miRNA, an aptamer, a CRISPR/Cas-based gene therapy agent, a peptide, a small molecule, a polymer, an expression vector encoding a gene of interest, or any combination thereof

In certain embodiments, the treatment and/or prophylactic methods may further comprising administering at least one more active agent. Such an agent may be an anti-inflammatory agent, an immunosuppressant, an anti-fibrotic agent, a vasodilator, and/or an analgesic. In certain embodiments, an agent that is used for treating SSc may be administered along with an agent or therapy that provides changes in gene expression or functions. For example, nintedanib, an NSAID, a corticosteroid, methotrexate, cyclosporine, anti-thymocyte globulin, mycophenolate mofetil and cyclophosphamide, a calcium channel blocker (e.g., nifedipine), an angiotensin converting enzyme inhibitor (ACE inhibitor), an endothelin-1 receptor inhibitor (e.g., bosentan), a prostaglandin (e.g., epoprostenol, prostacyclin), nitric oxide, and/or a collagen inhibitor (e.g., colchicine, para-aminobenzoic acid (PABA), dimethyl sulfoxide, and D-penicillamine) may be administered.

Applicant further discovered that the better therapeutic outcome of HSCT is associated with increased memory B cells, naïve B cells, and/or CD8+ T cells and reduced memory CD4+ T cells, resting CD4+ T cells, and/or naïve CD4+ T cells. Based on this, the method may further comprise increasing memory B cells, naïve B cells, and/or CD8+ T cells and/or reducing memory CD4+ T cells, resting CD4+ T cells, and/or naïve CD4+ T cells for treating or preventing SSc. This may be achieved by the active agent administered to a subject to alter expression or function of the gene product of one or more genes listed in FIG. 2A. Alternatively, it may be via administration of another active agent that provides such immune cell changes. The immune cell changes may be in to Ins of the absolute number of cells or percentage of cells. Changes in the gene expression and/or immune cells may be achieved in the blood or any body parts affected by SSc, which may be the skin, the vasculature, the gastrointestinal tract, the lung, the heart, and/or the kidney.

In further embodiments, the method further comprises detecting the expression or function of one or more of the genes the expression of which is to be increased or decreased, wherein said detecting occurs prior, during and/or after treatment.

### Therapeutic Effect Evaluation

The present discoveries in the gene expression changes in HSCT and differences between the HSCT and CYC treatments may be further applied to methods of evaluating therapeutic effects in a subject. Therefore, one aspect of the present invention relates to methods of determining whether a therapy for SSc is effective in a subject.

In some embodiments, the method may comprise measuring the expression of one or more genes listed in FIG. 2A in a sample from the subject before and at one or more time points after starting the therapy. If one or more genes listed in the second half of FIG. 2A (genes shown in FIG. 2A (continued)) are downregulated at least one time point after starting the therapy compared to before starting the therapy, the therapy may be determined effective. Such one or more genes may be one or more of ACVR1C, GREM2, NOG, ZFYVE9, NR3C2, LOC100131662, CFHR3, IL7R, and/or DNMT3A. Alternatively or additionally, if one or more genes listed in the first half of FIG. 2A are upregulated at least one time point after starting the therapy compared to before starting the therapy, the therapy may be determined effective. Such one or more genes may be one or more of E2F5, CD19, IL6, ZNF204P, and/or PLEKHF2.

In some embodiments, changes in immune cells may be quantified for evaluating therapeutic effects. An increase in memory B cells, naïve B cells, and/or CD8+ T cells may further support that the therapy is effective and a decrease in memory CD4+ T cells, resting CD4+ T cells, and/or naïve CD4+ T cells may further support that the therapy is effective. Conversely, an decrease in memory B cells, naïve B cells, and/or CD8+ T cells may support that the therapy is not effective and an increase in memory CD4+ T cells, resting CD4+ T cells, and/or naïve CD4+ T cells may further support that the therapy is not effective.

In evaluating the gene expression profile and/or immune cell quantities, a blood sample, a skin sample, a vascular sample, a gastrointestinal tract sample, a lung sample, a heart sample, and/or a kidney sample from the subject may be used. Preferably, a sample may be taken from the blood or the site that is affected by SSc.

### Therapeutic Agent Screening

The present discoveries in the gene expression changes in HSCT and differences between the HSCT and CYC treatments may be further applied to drug discovery methods. Therefore, one aspect of the present invention relates to methods of screening for a therapeutic agent for SSc.

In some embodiments, the method comprises (a) applying a candidate therapeutic agent to (I) one or more cells derived from a SSc patient, (II) one or more SSc cell line cells, or (III) a cell or tissue culture comprising a sample derived from a SSc patient; (b) after step (a), measuring the expression of one or more genes listed in FIG. 2A in (I) the one or more cells derived from a SSc patient, (II) the one or more SSc cell line cells, or (III) the cell or tissue culture comprising a sample derived from a SSc patient; (c) determining that a candidate therapeutic agent is effective if: (i) one or more genes listed in the second half of FIG. 2A (genes shown in FIG. 2A (continued)) are downregulated compared to an untreated or placebo control; and/or (ii) one or more genes listed in the first half of FIG. 2A are upregulated compared to an untreated or placebo control.

In some embodiments, downregulation of ACVR1C, GREM2, NOG, ZFYVE9, NR3C2, LOC100131662, CFHR3, IL7R, and/or DNMT3A may represent that the candidate agent is effective, and/or upregulation of E2F5, CD19, IL6, ZNF204P, and/or PLEKHF2 may represent that the candidate agent is effective. Conversely, upregulation of ACVR1C, GREM2, NOG, ZFYVE9, NR3C2, LOC100131662, CFHR3, IL7R, and/or DNMT3A may represent that the candidate agent is not effective, and/or downregulation of E2F5, CD19, IL6, ZNF204P, and/or PLEKHF2 may represent that the candidate agent is not effective.

In certain screening methods, the one or more cells in (I) or (II) comprise a skin cell (e.g., a keratinocyte), a fibroblast, a blood cell, an immune cell, a macrophage, a vascular ell, a gastrointestinal cell, a lung cell, a heart cell, and/or a renal cell. In yet certain methods, the cell or tissue culture in (III) comprises a skin tissue, an organoid, or a three-dimensional layered cell culture. In yet certain methods, the sample derived from a SSc patient in (III) comprises: (i) one or more fibroblasts derived from a SSc patient; (ii) one or more macrophages derived from a SSc patient; and/or (iii) serum or plasma derived from a SSc patient.

In particular embodiments, the cell or tissue culture in (III) is a three-dimensional, skin-like layered cell culture comprising: (i) one or more fibroblasts; and (ii) one or more keratinocytes; and optionally (iii) one or more monocytes or macrophages; and/or (iv) serum or plasma, wherein at least one of (i)-(iv) is derived from a SSc patient. In yet further embodiments, the cell or tissue culture in (III) may be Applicant's self-assembled skin equivalents (sSE) (Huang M. et al., Molecular Analysis of a Skin Equivalent Tissue Culture Model System of Systemic Sclerosis Using RNA Sequencing, Epigenetic Assays, Histology, and Immunoassays [abstract (Abstract Number 1903)]. Arthritis Rheumatol. 2018; 70 (suppl 10). https://acrabstracts.org/abstract/molecular-analysis-of-a-skin-equivalent-tissue-culture-model-system-of-systemic-sclerosis-using-ma-sequencing-epigenetic-assays-histology-and-immunoassays/. Accessed Sep. 15, 2020.).

### Therapy Response Prediction

The present discoveries in the gene expression changes in HSCT and differences between the HSCT and CYC treatments may be further applied to predicting responses to a SSc therapy. Therefore, one aspect of the present invention relates to methods of predicting whether a subject with SSc will respond to HSCT.

In some embodiments, the method comprises(a) measuring the expression of ACVR1C in a sample from the subject; and (b) determining that the subject will be a good responder to HSCT if the subject is a high expresser of ACVR1C. The correlation between decrease in ACVR1C and good response to HSCT was particularly evident in patients with SSc of the fibroproliferative subset. Therefore, optionally, the SSc the subject has may be fibroproliferative SSc.

Another aspect of the present invention relates to methods of predicting whether a subject with SSc will respond to CYC. (a) measuring the expression of CLCF1 in a sample from the subject; and (b) determining that the subject will be a good responder to CYC if the subject is a high expresser of CLCF1.

The prediction may be further applied to selection of the appropriate therapeutic method. Therefore, the present invention further encompasses methods of treating a subject having SSc. In some embodiments, the method may comprise (a) measuring the expression of ACVR1C in a sample from the subject; and (b) treating the subject with hematopoietic stem cell transplant (HSCT) if the subject is a high expresser of ACVR1C. Optionally, the SSc is fibroproliferative SSc. In some embodiments, the method may comprise(a) measuring the expression of CLCF1 in a sample from the subject; and (b) treating the subject with CYC if the subject is a high expresser of CLCF1.

The sample that may be used in measuring in the methods of predicting and methods of treating may optionally be a blood sample, a skin sample, a vascular sample, a gastrointestinal tract sample, a lung sample, a heart sample, and/or a kidney sample. In some embodiments, blood sample may be used. In some embodiments, a disease site or the part of the body affected by SSc may be used.

### Therapeutic and/or Prophylactic Agents

Based on Applicant's discoveries disclosed herein, also provided herein are agents for treating or preventing SSc.

In some embodiments, the agent for treating or preventing SSc may be: (i) one which decreases the expression of one or more genes listed in the second half of FIG. 2A (genes shown in FIG. 2A (continued)) (ii) one which suppresses, blocks, or inhibits the function of the gene product of one or more genes listed in the second half of FIG. 2A (genes shown in FIG. 2A (continued)); (iii) one which decreases the expression or function of ACVR1C, GREM2, NOG, ZFYVE9, NR3C2, LOC100131662, CFHR3, IL7R, and/or DNMT3A; (iv) one which increases the expression of one or more genes listed in the first half of FIG. 2A; (v) one which enhances the function of the gene product of one or more genes listed in the second half of FIG. 2A (genes shown in FIG. 2A (continued)); (vi) one which enhances the expression or function of E2F5, CD19, IL6, ZNF204P, and/or PLEKHF2; or any combination of the foregoing.

In some embodiments, such an agent optionally may be an antibody, an antigen-binding antibody fragment (e.g., scFv, Fab, Fab′, (Fab′)₂), a chimeric antigen receptor (CAR)-expressing cell, an siRNA, an shRNA, an miRNA, an aptamer, a

CRISPR/Cas-based gene therapy agent, a peptide, a small molecule, a polymer, an expression vector encoding a gene of interest, or any combination thereof.

In certain embodiments, the agent may comprise (i) a neutralizing, blocking, and/or antagonistic antibody or antigen-binding antibody fragment specific for ACVR1C, GREM2, NOG, ZFYVE9, NR3C2, LOC100131662, CFHR3, IL7R, and/or DNMT3A, (ii) a neutralizing, blocking, and/or antagonistic antibody or antigen-binding antibody fragment specific for ACVR1C, which optionally is designed to specifically or preferentially neutralize, block, and/or antagonize ACVR1C on macrophages, fibroblasts, and/or keratinocytes; (iii) an agonistic antibody or antigen-binding antibody fragment specific for E2F5, CD19, IL6, ZNF204P, and/or PLEKHF2 or any combination of any of the foregoing or a composition containing any of the foregoing.

In certain embodiments, such as agent may be contained in a composition, which further comprises a pharmaceutically acceptable carrier or excipient.

### Kits

Also provided herein are kits. In some embodiments, the kit may comprise: (a) at least one primer set for detecting expression of at least one gene listed in FIG. 2A; and (b) an instruction sheet. Such a kit may be used to measure expression of one or more genes listed in FIG. 2A, and may be useful in any of the inventive methods disclosed herein, for example, methods of determining whether a therapy for SSc is effective in a subject, methods of screening for a therapeutic agent for SSc, methods of predicting whether a subject with SSc will respond to HSCT or CYC, or methods of treating a subject with SSc.

In some embodiments, the kit may comprise: (a) (I) one or more cells derived from a SSc patient, (II) one or more SSc cell line cells, or (III) a cell or tissue culture comprising a sample derived from a SSc patient; and (b) at least one primer set for detecting expression of at least one gene listed in FIG. 2A. Optionally, the kit may for screening a therapeutic agent for treating SSC.

In certain kits, the one or more cells in (I) or (II) comprise a skin cell (e.g., a keratinocyte), a fibroblast, a blood cell, an immune cell, a macrophage, a vascular ell, a gastrointestinal cell, a lung cell, a heart cell, and/or a renal cell. In yet certain methods, the cell or tissue culture in (III) comprises a skin tissue, an organoid, or a three-dimensional layered cell culture. In yet certain methods, the sample derived from a SSc patient in (III) comprises: (i) one or more fibroblasts derived from a SSc patient; (ii) one or more macrophages derived from a SSc patient; and/or (iii) serum or plasma derived from a SSc patient.

In particular embodiments, the cell or tissue culture in (III) is a three-dimensional, skin-like layered cell culture comprising: (i) one or more fibroblasts; and (ii) one or more keratinocytes; and optionally (iii) one or more monocytes or macrophages; and/or (iv) serum or plasma, wherein at least one of (i)-(iv) is derived from a SSc patient. In yet further embodiments, the cell or tissue culture in (III) may be Applicant's self-assembled skin equivalents (sSE) (Huang M. et al., Molecular Analysis of a Skin Equivalent Tissue Culture Model System of Systemic Sclerosis Using RNA Sequencing, Epigenetic Assays, Histology, and Immunoassays [abstract (Abstract Number 1903)]. Arthritis Rheumatol. 2018; 70 (suppl 10). https://acrabstracts.org/abstract/molecular-analysis-of-a-skin-equivalent-tissue-culture-model-system-of-systemic-sclerosis-using-ma-sequencing-epigenetic-assays-histology-and-immunoassays/. Accessed Sep. 15, 2020.).

### Definitions

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims

All publications mentioned herein are incorporated herein by reference. Nothing herein is to be construed as an admission that the present invention is not entitled to antedate such disclosure by virtue of prior invention.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “about” or “approximately” as used herein when referring to a numerical value, such as of weight, mass, volume, concentration, or time, should not be limited to the recited numerical value but rather encompasses variations of +/−10% of a given value.

“ACVR1C” as used herein, also known as “activin A receptor type 1C”, “ALK7”, “ALK-7”, or “ACVRLK7”, is a type I receptor for the TGF beta family of signaling molecules. ACVR1C binds to activin A with low-moderate affinity and to activin B (Morianos I et al., J Autoimmun. 2019 November; 104: 102314. doi: 10.1016/j.jaut.2019.102314. Epub 2019 Aug 13. [PMID: 31416681]). ACVR1C forms a heteromeric complex with the ligand, which phosphorylates Smad2/3, which phosphorylates R-Smad, which, together with Smad4, suppresses the transcription factor NFkB and the downstream transcription. In humans, ACVR1C is encoded by the ACVR1C gene, located in Chromosome 2 (2q24.1). Different isoforms exist due to alternative splicing (Roberts H. J. et al., Biol Reprod. 2003 May; 68(5): 1719-26. doi: 10.1095/biolreprod.102.013045. Epub 2002 Dec. 27. [PMID: 12606401]), and the protein sequence may comprise, for example:

(SEQ ID NO: 1; 493 amino acids; NCBI Reference
Sequence: NP_660302.2)
MTRALCSALRQALLLLAAAAELSPGLKCVCLLCDSSNFTCQTEGACWAS
VMLTNGKEQVIKSCVSLPELNAQVFCHSSNNVTKTECCFTDFCNNITLH
LPTASPNAPKLGPMELAIIITVPVCLLSIAAMLTVWACQGRQCSYRKKK
RPNVEEPLSECNLVNAGKTLKDLIYDVTASGSGSGLPLLVQRTIARTIV
LQEIVGKGRFGEVWHGRWCGEDVAVKIFSSRDERSWFREAEIYQTVMLR
HENILGFIAADNKDNGTWTQLWLVSEYHEQGSLYDYLNRNIVTVAGMIK
LALSIASGLAHLHMEIVGTQGKPAIAHRDIKSKNILVKKCETCAIADLG
LAVKHDSILNTIDIPQNPKVGTKRYMAPEMLDDTMNVNIFESFKRADIY
SVGLVYWEIARRCSVGGIVEEYQLPYYDMVPSDPSIEEMRKVVCDQKFR
PSIPNQWQSCEALRVMGRIMRECWYANGAARLTALRIKKTISQLCVKED
CKA;
(SEQ ID NO: 2; 443 amino acids; NCBI Reference
Sequence: NP_001104501.1)
MLTNGKEQVIKSCVSLPELNAQVFCHSSNNVTKTECCFTDFCNNITLHL
PTASPNAPKLGPMELAIIITVPVCLLSIAAMLTVWACQGRQCSYRKKKR
PNVEEPLSECNLVNAGKTLKDLIYDVTASGSGSGLPLLVQRTIARTIVL
QEIVGKGRFGEVWHGRWCGEDVAVKIFSSRDERSWFREAEIYQTVMLRH
ENILGFIAADNKDNGTWTQLWLVSEYHEQGSLYDYLNRNIVTVAGMIKL
ALSIASGLAHLHMEIVGTQGKPAIAHRDIKSKNILVKKCETCAIADLGL
AVKHDSILNTIDIPQNPKVGTKRYMAPEMLDDTMNVNIFESFKRADIYS
VGLVYWEIARRCSVGGIVEEYQLPYYDMVPSDPSIEEMRKVVCDQKFRP
SIPNQWQSCEALRVMGRIMRECWYANGAARLTALRIKKTISQLCVKEDC
KA;
(SEQ ID NO: 3; 413 amino acids; NCBI Reference
Sequence: NP_001104502.1)
MTRALCSALRQALLLLAAAAELSPGLKCVCLLCDSSNFTCQTEGACWAS
VMLTNGKEQVIKSCVSLPELNAQVFCHSSNNVTKTECCFTDFCNNITLH
LPTGLPLLVQRTIARTIVLQEIVGKGRFGEVWHGRWCGEDVAVKIFSSR
DERSWFREAEIYQTVMLRHENILGFIAADNKDNGTWTQLWLVSEYHEQG
SLYDYLNRNIVTVAGMIKLALSIASGLAHLHMEIVGTQGKPAIAHRDIK
SKNILVKKCETCAIADLGLAVKHDSILNTIDIPQNPKVGTKRYMAPEML
DDTMNVNIFESFKRADIYSVGLVYWEIARRCSVGGIVEEYQLPYYDMVP
SDPSIEEMRKVVCDQKFRPSIPNQWQSCEALRVMGRIMRECWYANGAAR
LTALRIKKTISQLCVKEDCKA;
or
(SEQ ID NO: 4; 336 amino acids; NCBI Reference
Sequence: NP_001104502.1)
MTRALCSALRQALLLLAAAAELSPGLKCVCLLCDSSNFTCQTEGACWAS
VMLTNGKEQVIKSCVSLPELNAQVFCHSSNNVTKTECCFTDFCNNITLH
LPTDNGTWTQLWLVSEYHEQGSLYDYLNRNIVTVAGMIKLALSIASGLA
HLHMEIVGTQGKPAIAHRDIKSKNILVKKCETCAIADLGLAVKHDSILN
TIDIPQNPKVGTKRYMAPEMLDDTMNVNIFESFKRADIYSVGLVYWEIA
RRCSVGGIVEEYQLPYYDMVPSDPSIEEMRKVVCDQKFRPSIPNQWQSC
EALRVMGRIMRECWYANGAARLTALRIKKTISQLCVKEDCKA.

The full-length ACVR1C (SEQ ID NO: 1) has 493 amino acids and comprises an activin receptor-binding domain, a transmembrane domain, a GS domain, and a serine/threonine kinase domain (Roberts H. J. et al., Biol Reprod. 2003 May; 68(5): 1719-26. doi: 10.1095/biolreprod.102.013045. Epub 2002 Dec. 27. [PMID: 12606401]).

An “anti-ACVR1C agent” as used herein refers to any agents that are able to target ACVR1C directly or indirectly. Anti-ACVR1C agents of the present invention include, but are not limited to, anti-ACVR1C antibodies (Abs), anti-ACVR1C antigen-binding Ab fragments, anti-ACVR1C multi-specific Abs, anti-ACVR1C multi-specific antigen-binding Ab fragments, anti-ACVR1C ADCs, and anti-ACVR1C CARs. Anti-ACVR1C agents of the present invention further include an siRNA, shRNA, miRNA, and apatamer; and CRISPR/Cas-mediated gene targeting agent. In a broad sense, anti-ACVR1C agents may also encompass pharmaceutical compositions comprising any of the above-mentioned anti-ACVR1C agents.

The term “antibody” or “Ab,” or “immunoglobulin” is used herein in the broadest sense and encompasses various antibody structures which specifically binds with an antigen, including but not limited to monoclonal antibodies, polyclonal antibodies, multispecific antibodies (e.g., bispecific antibodies), and/or antibody fragments (also referred to as “antigen-binding antibody fragments”). Typically, a full-size Ab (also referred to as an intact Ab) comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Any isotype, such as IgG1, IgG2a, IgG2b, IgG3, IgG4, IgM, IgD, IgE, IgGA1, or IgGA2, may be used. Fc receptor types include, but are not limited to, FcaR (such as FcaRI), Fca/mR, FceR (such as FceRI, FceRII), FcgR (such as FcgRI, FcgRIIA, FcgRIIB1, FcgRIIB2, FcgRIIIA, FcgRIIIB), and FcRn and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is ACVR1C (also referred to as ALK7). Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

Certain amino acid modifications in the Fe region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half -life (Wang X. et al., Protein Cell. 2018 January; 9(1): 63-73; Dall'Acqua W. F. et al., J Biol Chem. 2006 Aug. 18; 281(33): 23514-24. Epub 2006 Jun. 21; Monnet C. et al, Front Immunol. 2015 Feb. 4; 6: 39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289(6): 3571-3590).

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, I332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F. et al., J Biol Chem. 2006 Aug. 18; 281(33): 23514-24. Epub 2006 Jun. 21; Wang X. et al., Protein Cell. 2018 January; 9(1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat).

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T, T256E, and/or any combination thereof (the residue numbering is according to the EU index as in Kabat). The Fc region optionally may further comprise one or more additional amino acid substitutions. Such substitutions may include but are not limited to M252Y, S254T, T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci U S A. 2017 Mar. 28; 114(13): 3485-3490. doi: 10.1073/pnas.1702173114. Epub 2017 Mar. 13).

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more potions or fragments of one or more Abs. An Ab fragment can be immunoreactive portions of intact immunoglobulins The term is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv. Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

The term “autoimmune disease” as used herein refers to a disease caused by the immune system that is dysregulated to attack healthy body tissues and/or cells of self Non-limiting examples of autoimmune disease include systemic sclerosis (SSc), ulcerative colitis (UC), Crohn's disease (CD), multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, and Chronic inflammatory demyelinating polyneuropathy (CIDP). Autoimmune diseases often cause fibrosis. For example, SSc can cause skin fibrosis, and SSc, RA, SLE, dermatomypsitis, psoriasis, and vasculitis can case interstitial lung disease (ILD) or pulmonary fibrosis (PF).

The term “fibrosis” as used herein refers to the condition describing formation or deposition of fibrous connective tissue, characterized by excess accumulation of extracellular matrix (ECM) such as collagen, in an organ or tissue. Fibrosis can severely disturb the function of such an organ or tissue. Exemplary fibrotic conditions and fibrotic diseases (i.e., diseases that cause fibrosis) are, but not limited to, scleroderma or systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), ulcerative colitis (UC), inflammatory bowel disease (IBD), Crohn's disease (CD), myelofibrosis, asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), myocardial fibrosis (e.g., interstitial fibrosis or replacement fibrosis), mediastinal fibrosis, retroperitoneal fibrosis, arterial stiffness, arthrofibrosis, Dupuytren's contracture, Peyronie's disease, and adhesive capsulitis. A disease that often result in fibrosis or a fibrotic condition is referred to herein as “fibrotic disease”. Fibrosis may be caused by autoimmune disease (as described above), infections (e.g., bacterial, viral such as hepatitis C, adenovirus, herpes virus), environmental factors (e.g., asbestos, grain dust, silica dust, radiation), medications (e.g., antibiotics, cardiac drugs, biologics), and/or genetic factors. The term “fibrogenesis” or “fibrogenic” refers to the mechanism and/or process of fibrosis formation.

The term “humanization” of an Ab refers to modification of an Ab of a non-human origin to increase the sequence similarity to an Ab naturally produced in humans. The term “humanized antibody” as used herein refers to Abs generated via humanization of an Ab. Generally, a humanized or engineered antibody has one or more amino acid residues from a source which is non-human, e.g., but not limited to mouse, rat, rabbit, non-human primate or other mammal. These human amino acid residues are often referred to as “import” residues, which are typically taken from an “import” variable, constant or other domain of a known human sequence. Known human Ig sequences are disclosed, e.g., www.ncbi.nlm.nih.gov/entrez/query.fcgi; www.atcc.org/phage/hdb.html, each entirely incorporated herein by reference. Such imported sequences can be used to reduce immunogenicity or reduce, enhance or modify binding, affinity, avidity, specificity, half-life, or any other suitable characteristic, as known in the art. Generally part or all of the non-human or human CDR sequences are maintained while part or all of the non-human sequences of the framework and/or constant regions are replaced with human or other amino acids. Antibodies can also optionally be humanized with retention of high affinity for the antigen and other favorable biological properties using three-dimensional immunoglobulin models that are known to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, framework (FR) residues can be selected and combined from the consensus and import sequences so that the desired antibody characteristic, such as increased affinity for the target antigen(s), is achieved. In general, the CDR residues are directly and most substantially involved in influencing antigen binding. Humanization or engineering of antibodies of the present invention can be performed using any known method, such as but not limited to those described in, for example, Winter (Jones et al., Nature 321: 522 (1986); Riechmann et al., Nature 332: 323 (1988); Verhoeyen et al., Science 239: 1534 (1988)), Sims et al., J Immunol. 151: 2296 (1993); Chothia and Lesk, J. Mol. Biol. 196: 901 (1987), Carter et al., Proc. Natl. Acad. Sci. U.S.A. 89: 4285 (1992); Presta et al., J. Immunol. 151: 2623 (1993), U.S. Pat. Nos. 5,723,323, 5,976,862, 5,824514, 5,817483, 5,814476, 5,763,192, 5,723,323, 5,766,886, 5,714,352, 6,187,287, 6,204,023, 6,180,370, 5,693,762, 5,530,101, 5,585,089, 5,225,539; 4,816,567, each entirely incorporated herein by reference, included references cited therein.

The term “inflammatory disease” or “inflammatory condition” refers to a disease or condition characterized by inflammation and include autoimmune diseases and allergies. Specific examples include but are not limited to SSc, inflammatory bowel disease, (IBD), ulcerative colitis (UC), Crohn's disease (CD), multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, and Chronic inflammatory demyelinating polyneuropathy (CIDP), asthma, nephritis, hepatitis, myosis, chronic peptic ulcer, periodontitis, perfusion injury, transplant rejection, pelvic inflammatory disease, Ankylosing Spondylitis (AS), Antiphospholipid Antibody Syndrome (APS), interstitial lung disease (ILD), pulmonary fibrosis (PF), idiopathic pulmonary fibrosis (IPF), rheumatoid arthritis (RA)-associated interstitial lung disease (RA-ILD), cystic fibrosis (CF), ulcerative colitis (UC), inflammatory bowel disease (IBD), Crohn's disease (CD), myelofibrosis, asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), myocardial fibrosis (e.g., interstitial fibrosis or replacement fibrosis), mediastinal fibrosis, retroperitoneal fibrosis, arterial stiffness, arthrofibrosis, Dupuytren's contracture, Peyronie's disease, and adhesive capsulitis. “Inflammatory disease” or “inflammatory condition” may further encompass diseases that cause or accompanied by neural inflammation, such as Alzheimer's disease (AD), Parkinson's disease (PD), multiple sclerosis (MS), motor neuron disease, ischemia, and traumatic brain injury, depression and autism.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

The term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “nucleic acid” and “polynucleotide” refer to RNA or DNA that is linear or branched, single or double stranded, or a hybrid thereof The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage. Excipients included in the formulations will have different purposes. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VII variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). Alternatively, the heavy chain variable domain may be placed downstream of the light chain variable domain, and the two domains may optionally be linked via a linker (for example, the G4S X3 linker).

The term “scleroderma” or “systemic sclerosis” as used herein refers to a group of autoimmune diseases that involve hardening and tightening of the skin and connective tissues. Fibrosis is the hallmark of the disease. Scleroderma may affect only the skin in some patients, but in many cases, also affect other parts of the body such as blood vessels, the digestive tract, and internal organs such as the heart, lungs, and kidneys.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The disease to be treated may be, for example, SSc, but may also treat other diseases that cause a similar condition and/or symptom to that of SSc. Therefore, the treatment method according to the present disclosure may also treat, a fibrotic disease, for example, pulmonary fibrosis, an interstitial lung disease, cystic fibrosis, chronic obstructive pulmonary disease, sarcoidosis, an allergic airway disease, hepatic fibrosis, or cardiac fibrosis. The condition to be treated by a method according to the present invention may be, for example, fibrosis, oxidative stress, or inflammation. In specific embodiments, the effect of the “treatment” may be evaluated by the amelioration of at least one measurable physical parameter of a disease, resulting from the administration of one or more therapies (e.g., anti-ACVR1C agent, and in some cases in combination with another therapy such as HSCT, CYC, or nintedanib). The parameter may be, for example, gene expression profiles, the mass of disease-affected tissues, inflammation-associated markers, fibrosis-associated markers, the number or frequency of disease-associated cells, the presence or absence of certain cytokines or chemokines or other disease-associated molecules, and may not necessarily discernible by the patient. In certain embodiments, the parameter may be event-free survival (EFS). In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of inflammatory or fibrotic tissue. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

Examples are provided below to illustrate the present invention. These examples are not meant to constrain the present invention to any particular application or theory of operation.

### EXAMPLES

### Example 1: Identification of Genes Associated With Successful Therapeutic Outcomes

The Scleroderma: Cyclophosphamide or Transplantation (SCOT) trial demonstrated that patients who underwent hematopoietic stem cell transplantation (HSCT) had better outcomes than those treated with cyclophosphamide (CYC) (Sullivan K. M. et al., N Engl J Med. 2018 Jan. 4; 378(1): 35-47. doi: 10.1056/nejmoa1703327. [PMID: 29298160]). Applicant subsequently demonstrated that patients who had the most significant difference in event free survival (EFS) were patients assigned to the fibroproliferative intrinsic subset using PBC samples (Franks J. M. et al., Ann Rheum Dis. 2020 Sep. 15; annrheumdis-2020-217033. doi: 10.1136/annrheumdis-2020-217033. Online ahead of print. [PMID: 32933919]]). Based thereon, Applicant investigated the mechanism by which HSCT resulted in improvement in SSc and the reason fibroproliferative patients showed the most significant response.

Briefly, Applicant systematically examined gene expression changes in the SCOT trial participants over time between the two treatment arms, CYC and HSCT. Differentially expressed genes were identified between treatment arms using linear mixed regression model and showed significant enrichment for TGFb signaling downregulation in the HSCT The non-canonical TGFb gene ACVR1C/ALK7 was most strongly associated with EFS in patients who underwent HSCT arm. By comparing the expression change of ACVR1C in SCOT to HSCT studies of other autoimmune diseases, Applicant found that ACVR1C has the largest fold-change in this study, suggesting that the changes may be specific to SSc. By creating the co-expression networks in “intrinsic” subsets, Applicant further found that the direct connections between ACVR1C and ZFYVE9 are variable across SSc intrinsic subsets and may be important for determining clinical response of SSc patients to HSCT.

### Methods

### Dataset

The SCOT trial compared the therapeutic outcomes of myeloablative CD34+selected autologous HSCT and CYC in severe diffuse SSc patients (for study design details of SCOT trial, see Sullivan K. M. et al., N Engl J Med. 2018 Jan. 4; 378(1): 35-47. doi: 10.1056/nejmoa1703327. [PMID: 29298160]). The trial provided gene expression in the peripheral blood cells (PBCs) from 30 SSc patients who completed HSCT treatment, 33 patients who completed CYC treatment, and 28 healthy controls (Franks J. M. et al., Ann Rheum Dis. 2020 Sep. 15; annrheumdis-2020-217033. doi: 10.1136/annrheumdis-2020-217033. Online ahead of print. [PMID: 32933919]). In the current study, Applicant analyzed the longitudinal expression profile of SCOT patients from baseline to 54 months. 20 month and 26 month data were merged (referred to as 20/26 months), and 48 month and 54 month data were merged (referred to as 48/54 months).

For studies on autoimmune diseases other than SSc, three independent longitudinal gene expression datasets of autoimmune diseases were analyzed. For Crohn's disease (CD), gene expression profiles for 18 CD patients receiving HSCT following for up to 52 weeks (GSE100922) derived from Corraliza et al. (Corraliza A. M. et al. J Crohns Colitis. 2019 Apr. 26; 13(5): 634-647. doi: 10.1093/ecco-jcc/jjy203. [PMID:30521002]) were analyzed. This dataset also provided labels for responder and non-responder. For multiple sclerosis (MS), gene expression data of CD4+ and CD8+ T cells from MS patients undergoing HSCT and during the 2 year follow up derived from De Paula A Souse A et al. (GSE32988) (De Paula A Souse A et al. Clin Sci (Lond). 2015 January; 128(2): 111-20. doi: 10.1042/CS20140095. [PMID:25116724]) were analyzed. To compare the non-SSc data with SCOT data, gene expression profiles from CD and MS were log-2 transformed and median centered if these steps were missing in the original datasets.

### Imputation for Missing Time Points in Longitudinal Data

In the SCOT trial, certain patients were not available for follow-up studies, e.g., those who had organ failure or died during the study (Sullivan K. M. et al., N Engl J Med. 2018 Jan. 4; 378(1): 35-47. doi: 10.1056/nejmoa1703327. [PMID: 29298160]), which resulted in non-random missing samples for patients who experienced events (See Results). To reduce the statistical bias of downstream comparisons, Applicant applied seasonal adjustment and linear interpolation to impute those missing time points. The “imputeTS” R package was used. Patients with only baseline gene expression data and no longitudinal samples were not imputed (n=20; 32% of participants; 10 for cyclophosphamide and 10 for transplant). To validate the imputation method, Applicant compared the results of the analysis using all participants and imputation of missing time points (n=63; 33 for cyclophosphamide and 30 for transplant) to the results obtained by analyzing only data for participants with complete data (n=20; 8 for cyclophosphamide and 12 for transplant) and to the results obtained analyzing all patient samples without any imputation (n=63; 33 for cyclophosphamide and 30 for transplant).

### Differentially Expressed Gene Identification and Pathway Enrichment

Linear mixed regression has been successfully fitted in another SSc clinical trial, ASSET, to identify the differences in longitudinal outcomes of patients treated with abatacept or placebo (Khanna D. et al., Arthritis Rheumatol. 2020 January; 72(1): 125-136. doi: 10.1002/art.41055. Epub 2019 Dec. 10. [PMID: 31342624]). In this study, to investigate the differences in gene expression between two treatment arms (HSCT vs CYC) in the SCOT longitudinal data, linear mixed regression was applied by adjusting for treatment, time point and patient age. Individual participants were considered as random effects. The “nlme” R package was used. The p value of the model was estimated via a one-way Analysis of Variance (ANOVA) test. False discovery rate (FDR) was calculated using p.adjust() R function. Genes with FDR<0.05 were considered as differentially expressed genes between two treatments. Pathway enrichment analysis was conducted using g:Profiler with default settings (Raudvere U. et al., Nucleic Acids Res. 2019 Jul. 2; 47(W1): W191-W198. doi: 10.1093/nar/gkz369. [PMID:31066453]). Adjusted p values were calculated with g:SCS and 0.05 was applied as the significance threshold for KEGG pathways.

### Cell Type Deconvolution

CIBERSORT (Chen B. et al., Methods Mol Biol. 2018; 1711: 243-259. doi: 10.1007/978-1-4939-7493-1_12. [PMID: 29344893]) was used to infer the proportions of 22 immune cell signatures using the imputed gene expression. Normalized enrichment scores were calculated using default settings and applied to linear mixed regression to identify the differences of immune cells between two treatments. Given the small pool, FDR<0.1 was used to consider the significance.

### Survival Analysis

Survival analysis was performed using the “survival” R package based on gene level data. Only the baseline gene expression data were considered for these analyses. Patients were divided into two groups using the median expression value of a given gene as cutoff Briefly, high expression group is patients' gene expression greater than or equal to the median value and low expression group is patients' gene expression lower than the median value. Log-rank test was applied to evaluate the differences of outcomes. Kaplan-Meier plot was used to visualize the results. The Cox proportional hazards model (Wald test) was applied to examine the association between a gene and patients' survival when considering additional clinical variables. Wilcoxon-test was applied to calculate p values for comparisons in boxplots.

### Co-Expression Network

To create the intrinsic subsets co-expression network based on TGF-beta genes, first correlations between the five TGF-beta genes and the remaining-expressed genes were calculated along with P values. Then, significant connections were selected out with p value <0.01 and correlations greater than 0.3 or lesser than −0.3. Cytoscape was used to plot the co-expression network using correlations as edges (Shannon P. et al., Genome Res. 2003 November; 13(11):2498-504. doi: 10.1101/gr.1239303. [PMID:14597658]).

### Results

### Validation of the Imputation Methods

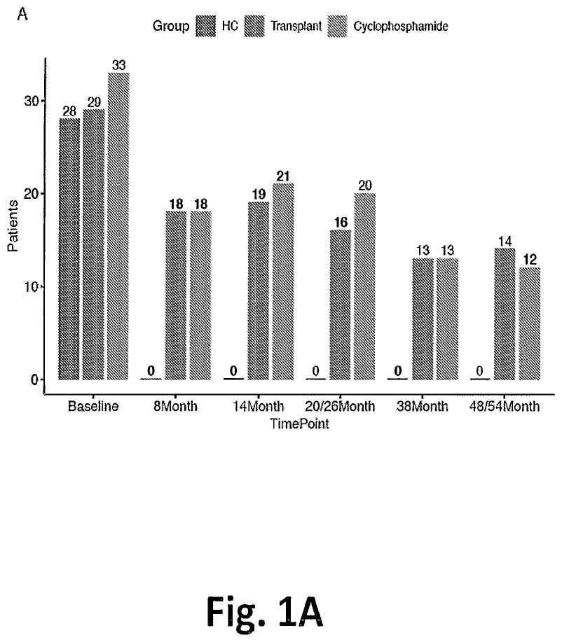

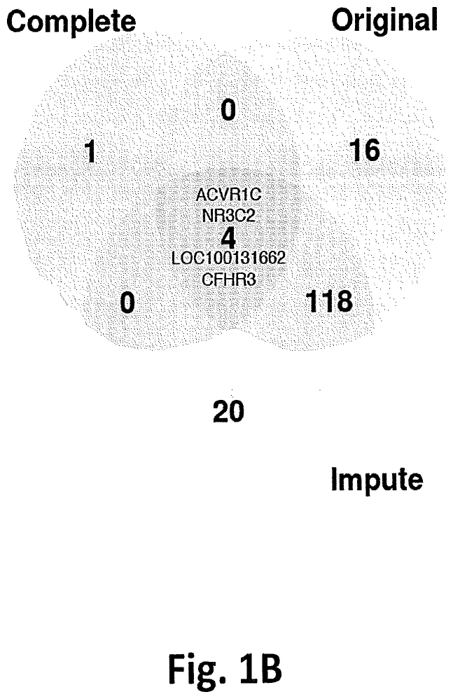

A number of participants in the SCOT study were missing time points due to non-random drop out due to either failure of EFS or death, resulting in non-random bias in the longitudinal gene expression data. Nearly half of the patients for each treatment lacked gene expression profiles for certain longitudinal time points (FIG. 1A). 12 patients in HSCT ani and 8 patients in CYC arm had complete gene expression data for all time points (baseline, 8 month, 14 month, 20/26 month, 38 month, and 48/54 month). To mitigate statistical bias for downstream comparisons, Applicant imputed time series data to estimate these data and reduce bias. In this study, seasonal adjustment and linear interpolation were applied to impute missing time points for each gene. A linear mixed regression algorithm was applied to investigate differentially expressed genes (FDR<0.05) between treatment arms using three different variants of the data: 1) using the patients with only complete data (referred to as “Complete”, n=20); 2) using the original data with missing time points (refer as “Original”, n=63); and 3) using the imputed data (refer as “Impute”, n=63).

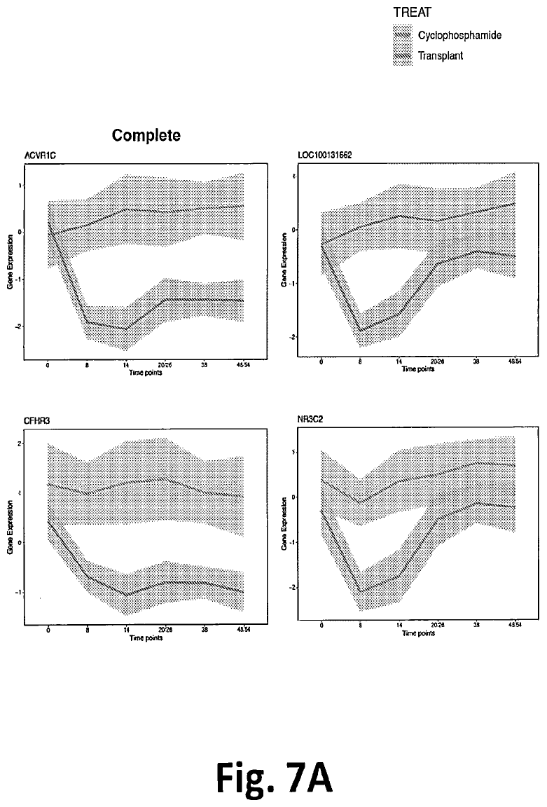

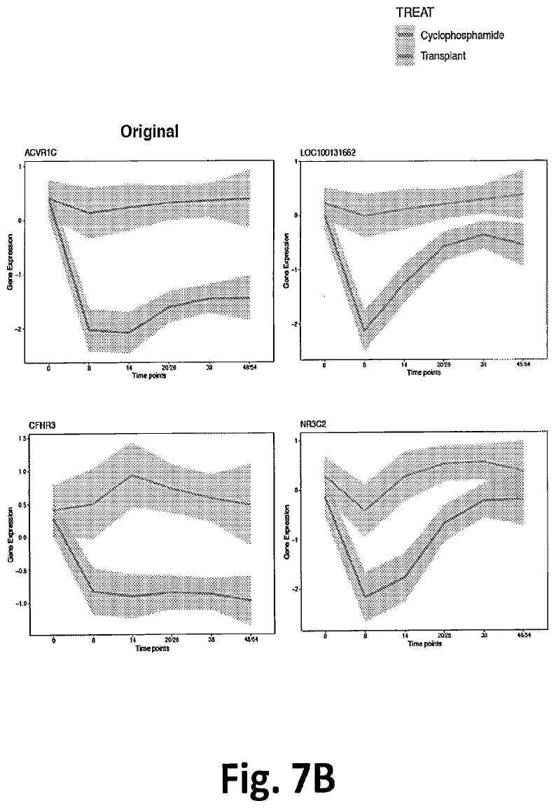

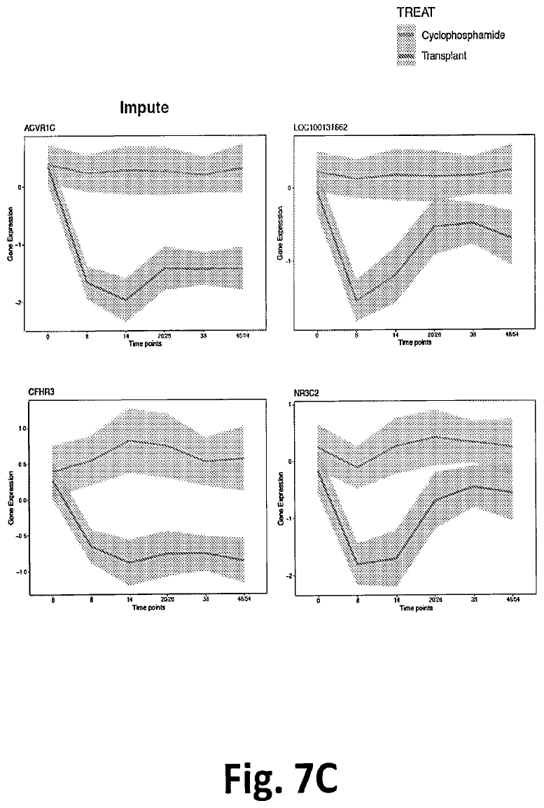

In the three data sets, Applicant identified 5, 138 and 142 differentially expressed genes between treatment arms, respectively (data not shown). 122 genes were overlapped between original and impute calculations (Jaccard index=0.77), and particularly four genes, ACVR1C, NR3C2, LOC100131662 and CFHR3, were shared by those three comparisons (FIG. 1B). The trends in the expression of the four genes between two treatments over time were highly consistent in the three comparisons (FIGS. 7A-7C). These observations suggest that using imputed gene expression profiles not only captures those most central genes but also identifies associated genes to provide more information. Therefore, in the following studies, Applicant focused on using the imputed gene expression for downstream analysis.

### Genes Differentially Expressed Between Treatment Arms

Using the imputed gene expression data of SCOT participants, 142 genes were identified to be significantly differentially expressed (FDR<0.05) between the two treatments (FIG. 1B). Expression changes over time for the 142 genes were analyzed (FIG. 2A).

A subset of genes (bottom cluster of the heatmap in FIG. 2A) had higher expression at baseline while their expression was decreased in the HSCT arm over time. These genes showed no significant changes over time and remained highly expressed in CYC compared to HSCT. One of these genes, ACV/JC, also known as ALK-7, is a type 1 receptor for the TGF-beta family of signaling molecules and interacts with SMAD2 (Asnaghi L. et al., Oncogene. 2019 March; 38(12): 2056-2075. doi: 10.10381s41388-018-0543-2. Epub 2018 Nov. 6. [PMID: 30401983]; Bondestam J. et al., Cytogenet Cell Genet. 2001; 95(3-4): 157-62. doi: 10.1159/000059339. [PMID: 12063393]). Its expression was significantly decreased in HSCT treatment compared to the flat trend in CYC (FIG. 2B; P=1.7e-09, FDR=2.6e-05). Similarly, the expression of interleukin-7 receptor (IL7R) dropped in HSCT compared to its expression in CYC (FIG. C; P=7.8e-06, FDR=0.006).

In contrast, the top cluster in FIG. 2A shows genes whose expression levels were significantly increased in HSCT compared CYC. For example, CD19, a marker for B cells, had increased expression in HSCT compared to CYC (FIG. 2D; P=3e-04, FDR=0.04). Interleukin 6 (IL6) also had a significant expression difference between treatment aims, while the expression was increased in both HSCT and CYC (FIG. 2E; P=2.5e-04, FDR=0.03).

### TGFb is a Central Downregulated Pathway in HSCT

Next, Applicant determined the pathways differentially expressed between treatment aims. Pathway enrichment analyses using the 142 differentially expressed genes were conducted. Five KEGG pathways were significantly enriched: the hematopoietic cell lineage; B cell receptor signaling; Epstein-Barr virus infection; TGF-beta signaling; and HIF-1 signaling pathways (adjusted P<0.05, data not shown).

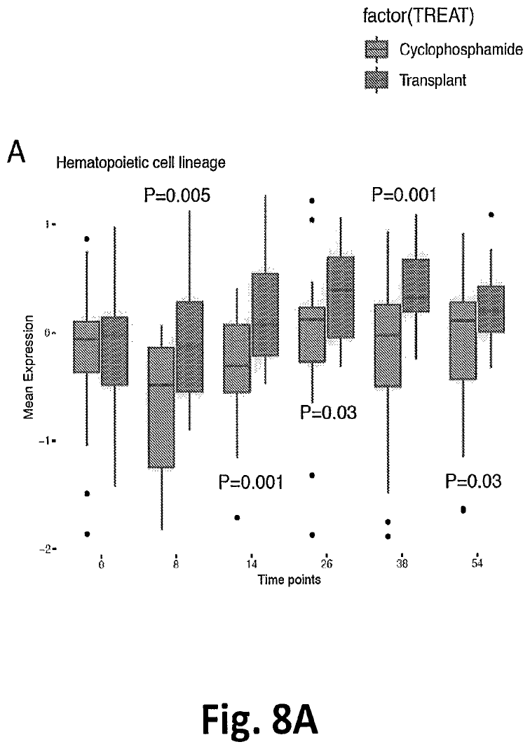

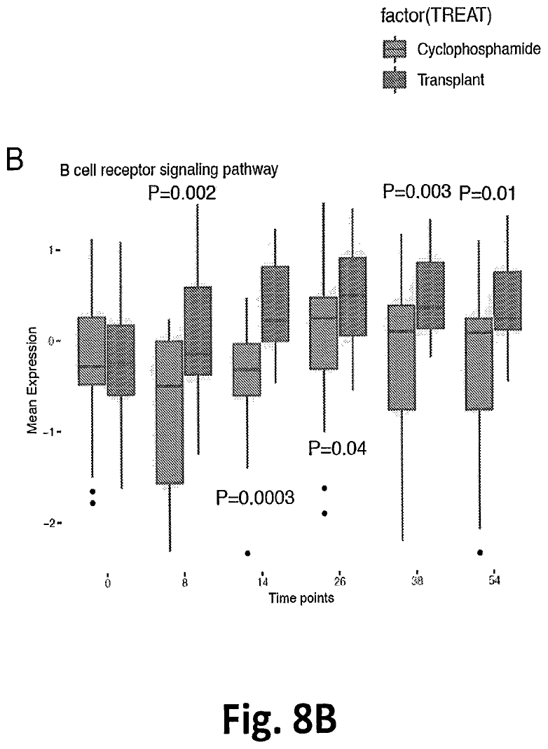

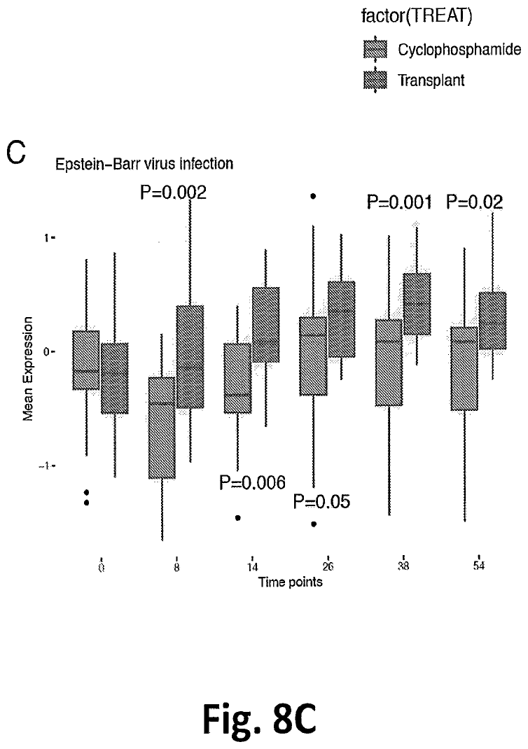

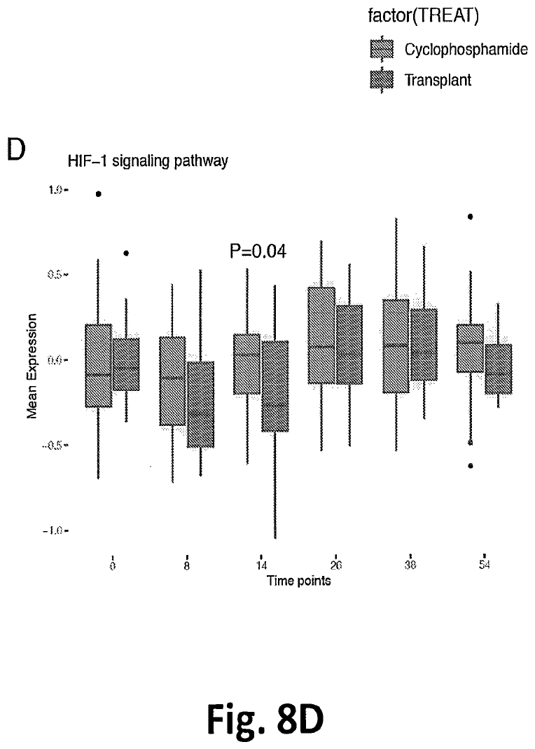

The average expression of the core enrichment genes for each pathway was used to compare pathway expression between treatment aims at each time point (FIG. 2F and FIGS. 8A-8D). The TGF-beta signaling pathway had the largest and most consistent differences at all post-treatment time points (FIG. 2F). Participants undergoing HSCT had significantly decreased TGF-beta signaling pathway compared to CYC (all P<5e-04).

Moreover, five out of the 142 genes were components of the TGF-beta signaling pathway (data not shown). These included the aforementioned ACVR1C (FIG. 2B), but also GREM2 (P=1.6e-08, FDR=1.2e-04), NOG (P=3.4e-04, FDR=0.04) and ZFYVE9 (P=4.7e-05, FDR=0.02), which showed similar trends between treatment arms (FIG. 2G-2I). E2F5 showed the opposite trend with increased expression in the HSCT arm (P=1.1e-04, FDR=0.02, FIG. 2J), which is consistent with its reported negative regulation of TGF-beta signaling (Xie L. et al., Breast Cancer Res. 2003; 5(6): R187-98. doi: 10.1186/bcr640. Epub 2003 Aug. 20. [PMID:14580254]). These results suggest that these non-canonical TGF-beta genes may play an important role in the biologic response of SSc patients to HSCT.

### B and T Cell Deconvolution Over Time in SCOT Participants

Applicant's hypothesis was that HSCT would reset the immune system in a way that does not occur during CYC treatment. To test this, Applicant inferred the relative proportion of immune cell-types with the imputed gene expression using CIBERSORT (Chen B. et al., Methods Mol Biol. 2018; 1711: 243-259. doi: 10.1007/978-1-4939-7493-1_12. [PMID: 29344893]).

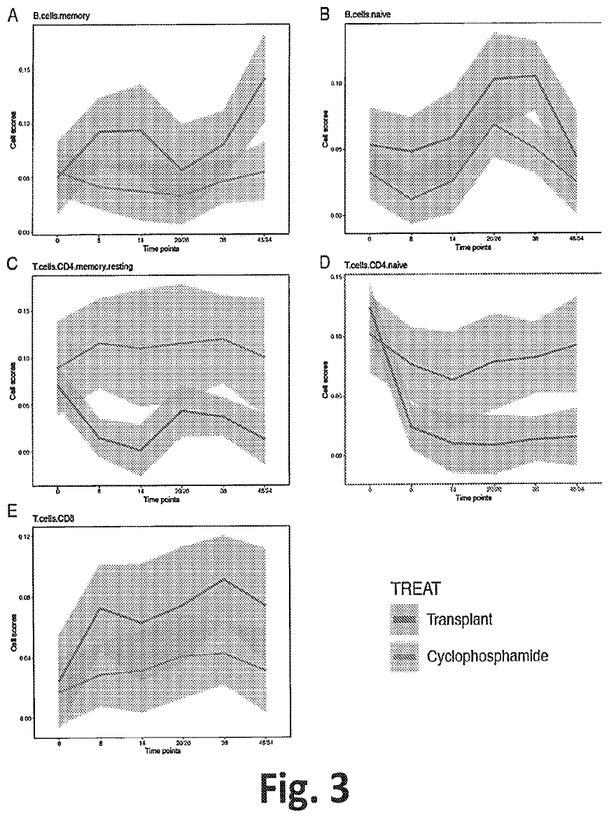

Major adaptive immune cell were in fact significantly affected. We found that memory B cells (P=0.02, FDR=0.09; FIG. 3A) and naïve B cells (P=0.008, FRD=0.06; FIG. 3B) had overall higher proportion in HSCT compared to CYC. These observations are in contrast with the trends of ACVR1C (FIG. 2B) or the TGF-beta pathway (FIG. 2F). This is in line with the negative regulation between TGF-beta and B cells (Tamayo E. et al., Int J Mol Sci. 2018 Dec. 7; 19(12): 3928. doi: 10.3390/ijms19123928. [PMID: 30544541]; Molnarfi N. et al., J Neuroinflammation. 2017 Jan. 19; 14(1): 13. doi: 10.1186/s12974-017-0798-5. [PMID: 28103949]; and Gros M. et al., J Immunol. 2008 Jun. 15; 180(12): 8153-8. doi: 10.4049/jimmuno1.180.12.8153. [PMID: 18523280]).

In contrast, we found the proportions of resting memory CD4+ T cells (P=0.009, FDR=0.06; FIG. 3C) and naïve CD4+ T cells (P=0.005, FDR=0.06; FIG. 3D) had significant decrease in HSCT compared to CYC, which follows the patterns of ACVR1C (FIG. 2B) and the TGF-beta pathway (FIG. 2F). These are consistent with the regulation between TGF-beta and T cell functions (Oh S. et al., J Immunol. 2013 Oct. 15; 191(8): 3973-9. doi: 10.4049/jimmuno1.1301843. [PMID: 24098055]; Li M. O. et al., Cell. 2008 Aug. 8; 134(3): 392-404. doi: 10.1016/j.ce11.2008.07.025. [PMID: 18692464]). However, we found that the pattern of CD8+ T cells (P=0.01, FRD=0.06; FIG. 3E) is more similar to that of B cells (FIG. 3A and B). This might suggest a negative association between CD8 T cells and TGF-beta (Tinoco R. et al., Immunity. 2009 Jul. 17; 31(1): 145-57. doi: 10.1016/j.immuni.2009.06.015. [PMID:19604493]).

### Baseline Expression of TGFb Genes Predicts Event Free Survival

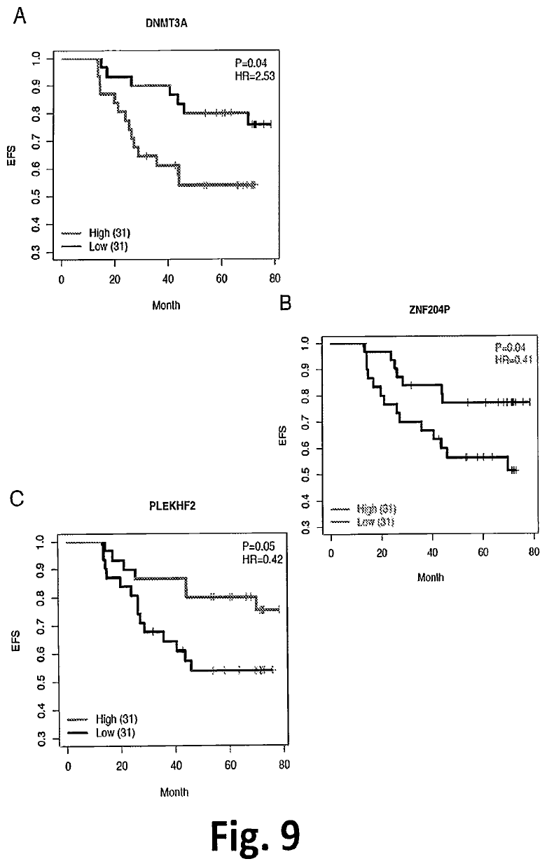

Leveraging EFS data, we asked whether the differentially expressed genes are able to predict EFS. We found that 3 out of 142 genes were significantly associated with EFS in all SCOT patients, when considering baseline gene expression levels, regardless of treatment. For example, SSc patients with lower DNA Methyltransferase 3 Alpha (DNMT3A) expression had increased EFS compared to patients with higher expression (P=0.04, HR=2.53, FIG. 9A). After adjusting for age and treatment, the expression of DNMT3A provided additional information about patient prognosis (P=0.053) from the multivariate Cox regression model, but the result did not reach significance. Previous studies have shown associations between DNMT3A and TGF-beta activity in multiple tissues (Koh H. B. et al., J Biol Chem. 2016 Sep. 9; 291(37): 19287-98. doi: 10.1074/jbc.M116.723080. Epub 2016 Jul. 12. [PMID: 27405758]; Thillainadesan G. et al., Mol Cell. 2012 Jun. 8; 46(5): 636-49. doi: 10.1016/j.molcel.2012.03.027. Epub 2012 May 3. [PMID: 22560925]; [PMID: 29727263]; Zhang Q. et al., PLoS One. 2011; 6(9): e25168. doi: 10.1371/journal.pone.0025168. Epub 2011 Sep. 30. [PMID: 21980391]). DNMT3A is associated with TGF-beta induced factor homebox 2 in the GIANT blood network with a score of 0.29 (seventh highest score) and TGF-beta induced factor homebox 1 in the GIANT skin fibroblast network with a score of 0.27 (eighteenth highest score) (data not shown) (Geene C. S. et al., Nat Genet. 2015 June; 47(6): 569-76. doi: 10.1038/ng.3259. Epub 2015 Apr. 27. [PMID: 25915600]). A higher calculated score reflects a stronger connection between two genes across multiple data types (Geene C. S. et al., Nat Genet. 2015 June; 47(6): 569-76. doi: 10.1038/ng.3259. Epub 2015 Apr. 27. [PMID: 25915600]). Additionally, two poorly studied genes, zinc finger protein 204 pseudogene (ZNF204P) and pleckstrin homology domain-containing family F member 2 (PLEKHF2), had the same trend, in which patients with higher expression had significantly better EFS than patients with lower expression (FIGS. 9B and 9C).

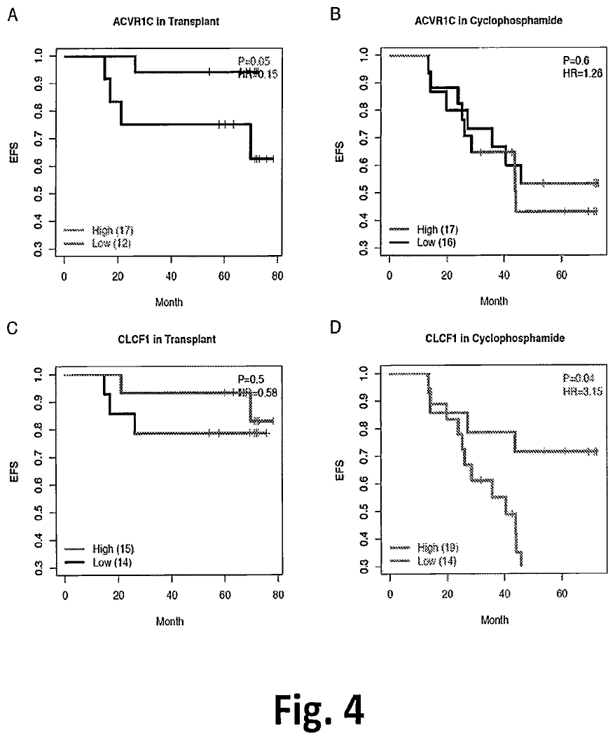

Fibroproliferative SSc participants were more likely to have EFS in HSCT relative to CYC (Franks J. M. et al., Ann Rheum Dis. 2020 Sep. 15; annrheumdis-2020-217033. doi: 10.1136/annrheumdis-2020-217033. Online ahead of print. [PMID: 32933919]) . ACVR1C is most highly expressed in the fibroproliferative subset of patients (see below). Moreover, we found these genes displayed distinct gene expression patterns in HSCT compared to CYC, and participants with differential expression had different EFS. Patients initially having higher ACVR1C expression had better EFS in HSCT at the end than patients initially having lower expression (P=0.05, HR=0.15, FIG. 4A). Eight of 17 patients with high ACVR1C expression were classified as fibroproliferative (Franks J. M. et al., Arthritis Rheumatol. 2019 October; 71(10): 1701-1710. doi: 10.1002/art.40898. Epub 2019 Sep. 2. [PMID: 30920766]), while only 3 patients with low ACVR1C expression were in this group. This suggest that pre-HSCT expression levels of ACVR1C might be a key determinant for eventual EFS. In contrast, the expression of ACVR1C alone does not predict EFS in the CYC arm (FIG. 4B).

In the CYC arm, cardiotrophin-like cytokine factor 1 (CLCF1) was the best predictor of EFS. Participants with higher CLCF1 expression had worse outcomes in CYC arm (P=0.04, HR=3.15, FIG. 4D), but no difference between outcomes in HSCT (FIG. 4C).

### Expression of ACVR1C in Other Autoimmune Diseases and HSCT Studies

In order to determine if these findings were specific to SSc participants or if this change in expression occurred in participants with other autoimmune diseases undergoing HSCT, we examined the expression change of ACVR1C in PBC studies of HSCT in Crohn's disease (CD) and multiple sclerosis (MS).

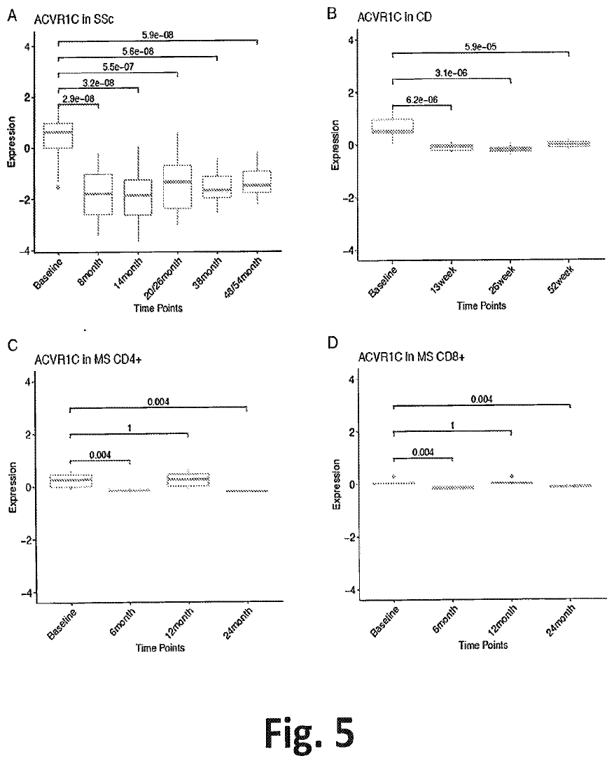

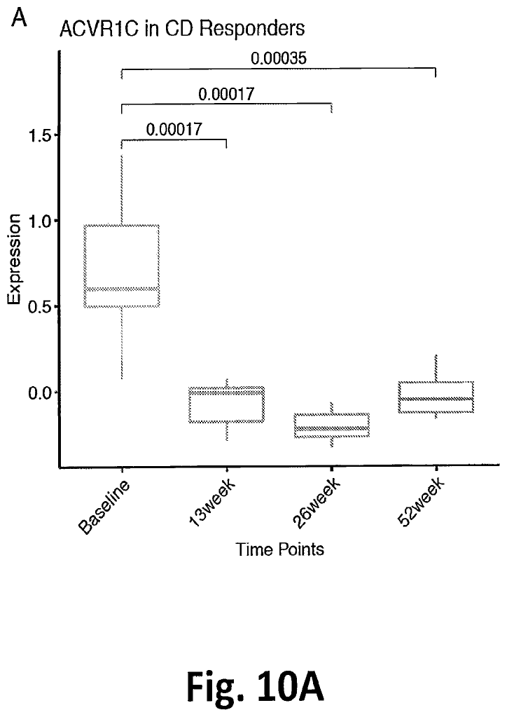

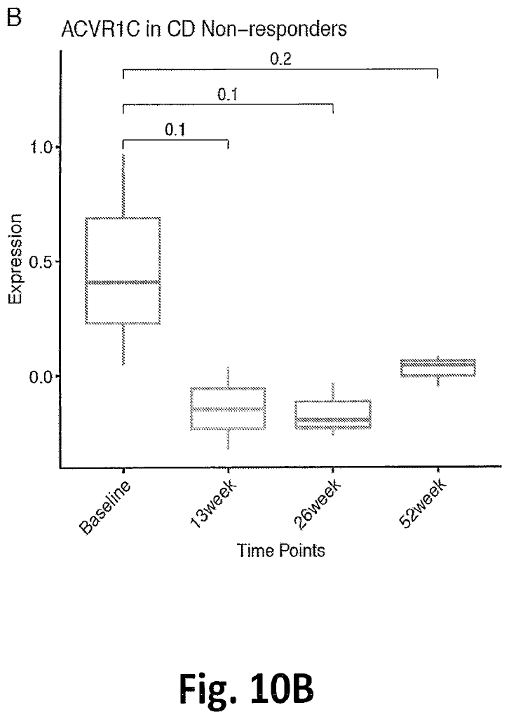

Analysis in the CD dataset (Corraliza A. M. et al., J Crohns Colitis. 2019 Apr. 26; 13(5): 634-647. doi: 10.1093/ecco-jcc/jjy203. [PMID: 30521002]) showed that ACVR1C expression was significantly decreased with HSCT treatment, but was relatively flat over time reflecting the small fold-change of 0.86 (FIG. 5B). This trend was driven by those participants who responded to treatment, as defined in the original paper (FIGS. 10A-10B). Similarly, in MS (De Paula A Souse A et al. Clin Sci (Lond). 2015 January; 128(2): 111-20. doi: 10.1042/CS20140095. [PMID: 25116724]), we found a significant decrease in ACVR1C expression post-treatment (baseline vs 6 month time point) but expression was relatively unchanged across CD4+ and CD8+ in MS patients over time (FIG. 5C and D). Neither the CD study nor the MS reported ACVR1C as the differentially expressed genes due to the low fold-changes of the gene (0.86-, 0.83- and 2.2- fold decrease in CD, CD4+ MS, and CD8+ MS respectively). However, in SCOT, ACVR1C had a 3.6-fold decrease between the last time point and baseline.

### ACVR1C Network in the Fibroproliferative Subset

In order to determine if ACVR1C expression was decreased across intrinsic subsets in SCOT participants, we performed the linear mixed regression between the two treatment arms for each intrinsic subset.

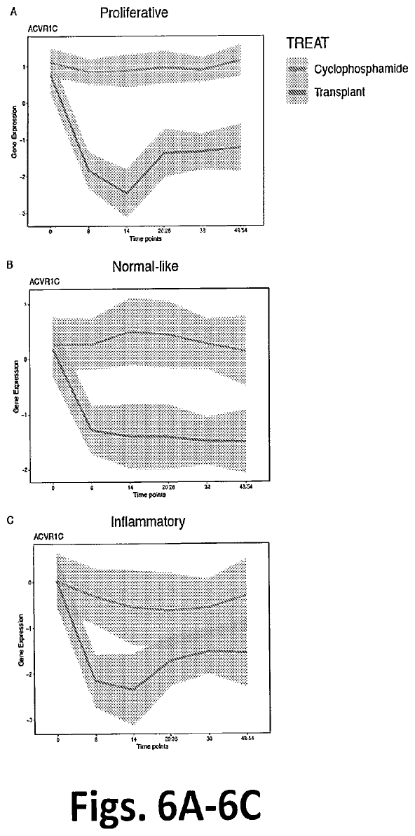

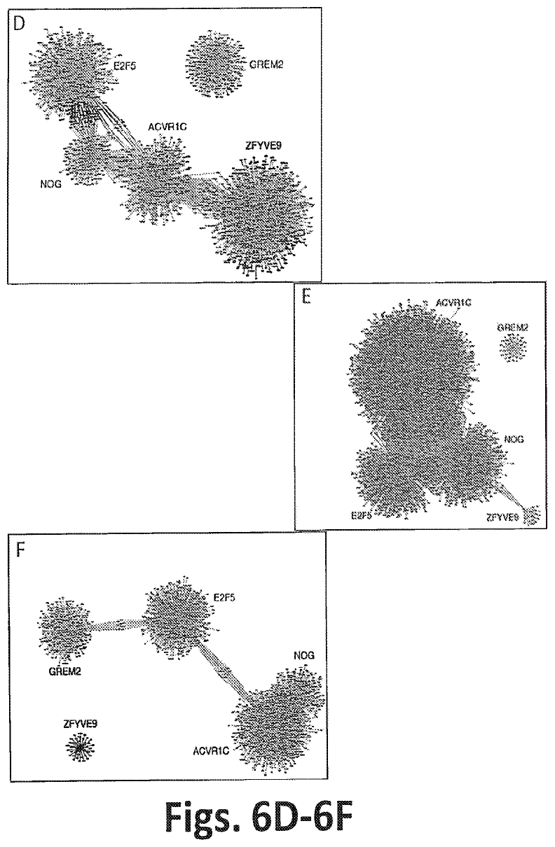

We identified 69 differentially expressed genes (FDR<0.05) in the fibroproliferative subset and 16 differentially expressed genes in the normal-like subset (data not shown). Nine genes were shared between these two gene sets. There were no significant differentially expressed genes identified for the inflammatory subset between treatment arms. ACVR1C was the most significant gene in both the fibroproliferative (P=1.1e-07,FDR=0.002) and normal-like (P=3.1e-07, FDR=0.005) subsets (FIG. 6A and 6B). Although ACVR1C expression initially decreases in patients of the inflammatory subset in the HSCT arm (FIG. 6C), a rebound after 14 months, coupled with higher variation of expression in both treatment arms, results in non-significant results (P=0.05, FDR=0.9).

Fibroproliferative and normal-like subsets displayed similar ACVR1C expression changes, significant decreases in HSCT compared to CYC (FIG. 6A and 6B). However, Franks et al. identified that EFS was very distinct between fibroproliferative and normal-like patients undergoing HSCT (Franks J. M. et al., Ann Rheum Dis. 2020 Sep. 15; annrheumdis-2020-217033. doi: 10.1136/annrheumdis-2020-217033. Online ahead of print. [PMID: 32933919]) . We therefore wanted to pursue the molecular underpinning of this difference. We tested two hypotheses. First, that the fibroproliferative patients have higher baseline ACVR1C expression compared to inflammatory and normal-like patients, because we showed that patients with high ACVR1C expression had better outcomes (FIG. 4A). Second, that the difference reflects different gene-gene network connectivity between the intrinsic subsets.

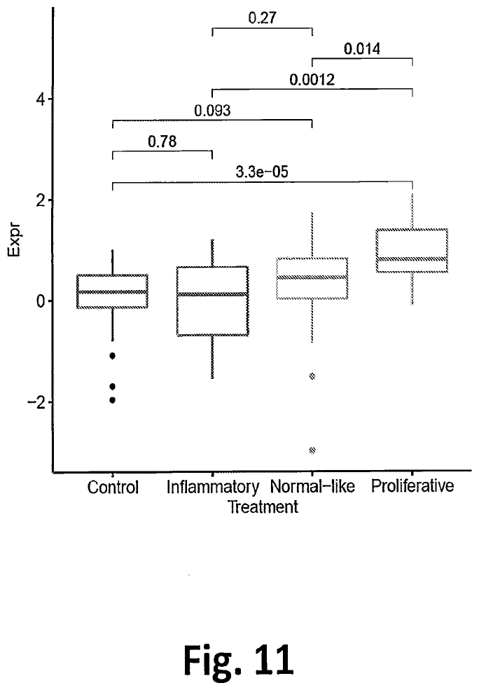

To test these hypotheses, we first compared the expression of ACVR1C between intrinsic subsets and healthy controls in SCOT (FIG. 11). Fibroproliferative patients had the highest expression of ACVR1C compared to inflammatory (P=0.001), normal-like (P=0.01) and healthy controls (P=3e-05). Additionally, ACVR1C expression was not significantly different between inflammatory, normal-like, and healthy controls.

To determine if there are any changes in the gene-gene network connectivity, we examined the co-expression network between the 5 TGF-beta genes and the rest of the genome. The network was filtered using only significant connections (absolute correlation >0.3 and P<0.01). In the co-expression network of fibroproliferative patients (FIG. 6D), ACVR1C was directly connected to E2F5, NOG, and ZFYVE9 and GREM2 gene cluster was isolated. While in the normal-like co-expression network (FIG. 6E), ACVR1C and ZFYVE9 were connected to the NOG cluster separately. The inflammatory network showed a completely different pattern with GREM2 connected to E2F5, and ZFYVE9 being completely isolated (FIG. 6F). Those observations suggest that the co-expressions networks of ACVR1C and ZFYVE9 clusters are different across intrinsic subsets and may affect the response to treatment in SSc patients.

### Discussion

Missing data are inevitable in longitudinal clinical trials and variety of algorithms have been developed to deal with the missing data (Dziura J. D. et al., Yale J Biol Med. 2013 Sep. 20; 86(3): 343-58. eCollection 2013 September. [PMID: 24058309]). However, selecting an appropriate method for a given clinical trial is challenging (Little R. J. et al., N Engl J Med. 2012 Oct. 4; 367(14): 1355-60. doi: 10.1056/NEJMsr1203730. [PMID: 23034025]). For the SCOT trial, missing clinic visits were caused by death or internal organ (lung or renal) failure during the trial (FIG. 1A). Therefore, methods for dealing with data Missing at Random (MAR) or Missing Not at Random (MNAR) should be considered to apply to this gene expression profile. In this study, to reduce the downstream statistical analysis bias caused by those missing time points, our strategy was (1) to impute the missing time points using seasonal adjustment and linear interpolation algorithm and (2) to investigate the differences between two treatments using linear mixed regression model. Here, we assumed that the longitudinal gene expression profile is similar to time series data.

To validate the imputation methods, we identified the differentially expressed genes between two arms by integrating linear mixed regression model and gene expression profiles containing (1) patients with no missing data, (2) all available patients and (3) all patients with the imputation of missing time points. We found that using the imputed gene expression data, the results were highly consistent with using it from all available patients (FIG. 1B, Jaccard index=0.77). Four genes were overlapped among the comparisons and their differences between two treatments were very similar along with time points across comparisons (FIG. 7A-7C). Moreover, the most key finding, ACVR1C, displayed a consistency in the samples without missing data, in all patients and when using imputation. This suggests that using the imputation, we don't lose the real differential genes but providing more potential candidates for downstream analysis.

We found that HSCT significantly decreased ACVR1C expression in the SCOT patients compared to CYC (FIG. 2B) and participants with high ACVR1C expression had significantly better outcomes than those with low expression (FIG. 4A). Fibroproliferative patients had the highest expression of ACVR1 C which may explain why fibroproliferative patients had better EFS in HSCT compared to inflammatory and normal-like patients (FIG. 11). TGFb, which plays an important role in mediating fibrogenesis, has long been implicated in SSc as a potential driver of pathogenesis and also as therapeutic target (Varga J. et al., Nat Rev Rheumatol. 2009 April; 5(4): 200-6. doi: 10.1038/nrrheum.2009.26. [PMID: 19337284]). A trial of the TGFb inhibitor fresoliumab showed promising results (Rice L. M. et al., J Clin Invest. 2015 Jul. 1; 125(7): 2795-807. doi: 10.1172/JCI77958. Epub 2015 Jun. 22. [PMID: 26098215]) suggesting changes in this pathway may be connected to patient improvement. TGF-beta plays a diversity of roles in regulating the immune system (Sanjabi S. et al., Cold Spring Harb Perspect Biol. 2017 Jun. 1; 9(6): a022236. [PMID: 28108486]). In this study, we found that the decreased TGF-beta pathway caused by HSCT in SSc might negatively regulate B and CD8 T cells and positively regulate CD4 T cells (FIG.

3).

ACVR1C showed small changes in expression levels in patients with other autoimmune diseases undergoing HSCT (FIG. 5). Among HSCT datasets, we found that the expression of ACVR1C has the largest fold decrease in SSc compared to other two autoimmune diseases (FIG. 5). This result suggests a potentially important role of ACVR1C in determining the response to HSCT. Four related TGFb signaling genes, E2F5, NOG, ZFYVE9 and GREM2, also showed significantly differences between treatment arms, which suggests that decreased TGFb signaling in PBCs might be a key component for reducing the development of the disease (FIG. 3A). Moreover, compared to canonical TGFb genes, these non-canonical genes might also play important roles in determining and affecting the disease which provides novel insights of genes for this disease.

In conclusion, Applicant found a strong association between non-canonical TGFb genes and HSCT in SSc. The results suggest that (i) ACVR1C is an effective target for treating and/or preventing SSc, (ii) increased expression of ACVR1C provides may be a marker for diagnosing SSc or an indication that the patient will respond to HSCT, (iii) decreased expression of ACVR1C is a good indicator for determining that a patient is responding to HSCT and/or another therapy, or that a subject candidate therapeutic agent is effective for treating or preventing SSc, (iv) an agent that reduces the expression or function of ACVR1C may be an effective therapeutic or prophylactic agent for SSc. Any one of the genes identified through this study may be further targeted or used as an indicator. Since SSc is a fibrotic disease, the present findings may well be further applicable to other fibrotic diseases. Since SSc is an autoimmune and inflammatory disease, the findings may also be applied to other autoimmune and/or inflammatory diseases.

What is claimed is:

1. A method of treating or preventing a fibrotic, autoimmune, and/or inflammatory disease or condition in a subject, comprising treating a subject in need thereof with an active agent that reduces the expression or function of ACVR1C,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjögren syndrome, Guillain-Barre syndrome, or Chronic inflammatory demyelinating polyneuropathy (CIDP),
further optionally wherein the fibrotic, autoimmune, and/or inflammatory disease or condition is SSc, optionally wherein the subject has been determined to overexpress ACVR1C prior to and/or during treatment.

2. A method of treating or preventing a fibrotic, autoimmune, and/or inflammatory disease or condition in a subject, comprising treating a subject in need thereof with an active agent that alters the expression or function of one or more genes listed in FIG. 2A (NOG, ACVR1C, SOX8, GREM2, DPP4, SATB1-AS1, IL7R, DSEL, RNF157, ZFYVE9, EXOC8, TCEAL6, RAB43, ZNF217, HERPUD2, ZNF33A, PDCD4-AS1, CREBRF, PRKAG2, INADL, TCEAL2, TSPEAR-AS2, GPR155, RNF144A, SERINC5, SATB1, EDA, LMTK3, DHRS3, ZNF658, PDE3B, TSPAN18, TLE2, AKT3, CDKN1B, DNMT3A, CEP170B, TOB1, CYTIP, DLGAP4, D2HGDH, KIF9-AS1, ARHGEF4, TTC39B, NR3C2, PLEKHB1, EPHX2, KDF1, LOC100131662, PLAG1, ZNF204P, FAM153B, PLCL1, CACNA1I, BEX5, TRPC1, MAGI3, EPHA1, ISM1, ATP6V0E2-AS1, TMEM30B, CFH, CFHR3, EXPH5, STX17-AS1, PRKCA, XLOC_I2_010062, KIF5C, FAM134B, CMTMB, ITGA6, TRABD2A, MAL, NEFL, or TCEA3, Inc-STEAP1B-1, P2RX5, FCRL2, FFAR1, PPAPDC1B, TCF4, IL7, RRAS2, LOC100131043, PKIG, MARCH3, CXXC5, BARD1, LINC00926, TSPAN13, SLC17A9, CHST10, RHOBTB2, CYB561A3, CLCF1, SYVN1, PEAK1, DUS2, ATP5B, DEF8, FADS3, STX18, HVCN1, UCP2, PMEPA1, CD83, CRIP3, C150C15ORF57, 57, LDHA, EAF2, SNX29, BCL11A, NT5DC2, PLEKHF2, BLNK, BLK, PNOC, MS4A1, SNX22, LOC100130458, FCRLA, PCDH9, CR2, OSBPL10, CDCA7, E2F5, HS3ST1, PLEKHG1, EBF1, ABCB4, BEND4, POU2AF1, CD200, LOC101059954, NCALD, EOMES, HLA-DOB, CD19, TRAF4, CORO2B, IL6, or CD72, or any combination thereof),
optionally wherein at least the expression or function of ACVR1C is reduced, further optionally wherein the subject has been determined to overexpress ACVR1C prior to and/or during treatment,
still further optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Bane syndrome, or Chronic inflammatory demyelinating polyneuropathy (CIDP),
and further optionally wherein the fibrotic, autoimmune, and/or inflammatory disease or condition is SSc.

3. The method according to claim 2, wherein the altering comprises reducing the expression or function of one or more genes listed in the second half of FIG. 2A (genes shown in FIG. 2A (continued) (NOG, ACVR1C, SOX8, GREM2, DPP4, SATB1-AS1, IL7R, DSEL, RNF157, ZFYVE9, EXOC8, TCEAL6, RAB43, ZNF217, HERPUD2, ZNF33A, PDCD4-AS1, CREBRF, PRKAG2, INADL, TCEAL2, TSPEAR-AS2, GPR155, RNF144A, SERNIC5, SATB1, EDA, LMTK3, DHRS3, ZNF658, PDE3B, TSPAN18, TLE2, AKT3, CDKN1B, DNMT3A, CEP170B, TOB1, CYTIP, DLGAP4, D2HGDH, KIF9-AS1, ARHGEF4, TTC39B, NR3C2, PLEKHB1, EPHX2, KDF1, LOC100131662, PLAG1, ZNF204P, FAM153B, PLCL1, CACNA1I, BEX5, TRPC1, MAGI3, EPHA1, ISM1, ATP6V0E2-AS1, TMEM30B, CFH, CFHR3, EXPH5, STX17-AS1, PRKCA, XLOC_I2_010062, KIF5C, FAM134B, CMTMB, ITGA6, TRABD2A, MAL, NEFL, or TCEA3, or any combination thereof), optionally wherein at least the expression or function of ACVR1C is reduced, further optionally wherein the expression or function of NR3C2, LOC100131662 CFHR3 ACVR1C, GREM2, NOG, ZFYVE9 ACVR1C IL7R and/or DNMT3A is reduced, still further , optionally wherein the subject has been determined to overexpress ACVR1C prior to and/or during treatment.

4. The method according to claim 1, 2 or 3, which comprises increasing or enhancing the expression or function of one or more genes listed in the first half of FIG. 2A (Inc-STEAP1B-1, P2RX5, FCRL2, FFAR1, PPAPDC1B, TCF4, IL7, RRAS2, LOC100131043, PKIG, MARCH3, CXXC5, BARD1, LINC00926, TSPAN13, SLC17A9, CHST10, RHOBTB2, CYB561A3, CLCF1, SYVN1, PEAK1, DUS2, ATP5B, DEF8, FADS3, STX18, HVCN1, UCP2, PMEPA1, CD83, CRIP3, C150C15ORF57, 57, LDHA, EAF2, SNX29, BCL11A, NT5DC2, PLEKHF2, BLNK, BLK, PNOC, MS4A1, SNX22, LOC100130458, FCRLA, PCDH9, CR2, OSBPL10, CDCA7, E2F5, HS3ST1, PLEKHG1, EBF1, ABCB4, BEND4, POU2AF1, CD200, LOC101059954, NCALD, EOMES, HLA-DOB, CD19, TRAF4, CORO2B, IL6, or CD72, or any combination thereof).

5. The method according to claim 1, 2, 3, or 4, which comprises increasing the expression or function of E2F5, CD19, IL6, ZNF204P, and/or PLEKHF2.

6. The method according to any one of claims 1-5, comprising administering hematopoietic stem cell transplantation (HSCT) to the subject, optionally thereby further reducing or increasing the expression or function of said one or more genes, further optionally thereby further modifying the expression or function of one or more genes of the TGF-beta, BMP, and/or ACVR1C signaling pathway in the treated subject.

7. The method according to any of claims 1-6, which comprises altering the expression or function of one or more genes of the TGF-beta, BMP, and/or ACVR1C signaling pathway.

8. The method according to any of claims 1-7, comprising one or more of the following:
(i) decreasing the expression or function of ACVR1C;
(ii) decreasing the expression or function of GREM2;
(iii) decreasing the expression or function of NOG;
(iv) decreasing the expression or function of ZFYVE9; and/or
(v) increasing the expression or function of E2F5.

9. The method according to any of claims 1-7, comprising one or more of the following:
(i) decreasing the expression or function of ACVR1C;
(ii) decreasing the expression or function of GREM2;
(iii) decreasing the expression or function of NOG;
(iv) decreasing the expression or function of ZFYVE9;
(v) increasing the expression or function of E2F5;
(vi) decreasing the expression or function of NR3C2;
(vii) decreasing the expression or function of LOC100131662;
(viii) decreasing the expression or function of CFHR3;
(ix) decreasing the expression or function of IL7R;
(x) increasing the expression or function of CD19;
(xi) increasing the expression or function of IL6;
(xii) decreasing the expression or function of DNMT3A;
(xiii) increasing the expression or function of ZNF204P; and/or
(xiv) increasing the expression or function of PLEKHF2.

10. The method according to any of claims 1-9, which comprises administering to the subject:
(a) an agent that decreases the expression or function of ACVR1C, GREM2, NOG, ZFYVE9, NR3C2, LOC100131662, CFHR3, IL7R, and/or DNMT3A; and/or
(b) an agent that increases the expression or function of E2F5, CD19, IL6, ZNF204P, and/or PLEKHF2.

11. The method according to any of claims 1-10, which comprises administering to the subject a composition comprising:
(a) an agent that decreases the expression or function of ACVR1C, GREM2, NOG, ZFYVE9, NR3C2, LOC100131662, CFHR3, IL7R, and/or DNMT3A; and/or
(b) an agent that increases the expression or function of E2F5, CD19, IL6, ZNF204P, and/or PLEKHF2.

12. The method according to any of claims 1-11, wherein the agent which reduces or increases gene expression or function is an antibody, an antigen-binding antibody fragment (e.g., scFv, Fab, Fab′, (Fab′)₂), a chimeric antigen receptor (CAR)-expressing cell, an siRNA, an shRNA, an miRNA, an aptamer, a CRISPR/Cas-based gene therapy agent, a peptide, a small molecule, a polymer, an expression vector encoding a gene of interest, or any combination thereof.

13. The method according to any one of claims 1-12, further comprising administering at least one other active agent.

14. The method according to claim 13, wherein the other active agent is an anti-inflammatory agent, an immunosuppressant, an anti-fibrotic agent, a vasodilator, and/or an analgesic.

15. The method according to claim 13 or 14, wherein the at least one other active agent is selected from nintedanib, an NSAID, a corticosteroid, methotrexate, cyclosporine, anti-thymocyte globulin, mycophenolate mofetil and cyclophosphamide, a calcium channel blocker (e.g., nifedipine), an angiotensin converting enzyme inhibitor (ACE inhibitor), an endothelin-1 receptor inhibitor (e.g, bosentan), a prostaglandin (e.g., epoprostenol, prostacyclin), nitric oxide, and/or a collagen inhibitor (e.g., colchicine, para-aminobenzoic acid (PABA), dimethyl sulfoxide, and D-penicillamine).

16. The method according to any one of claims 1-15, further comprising
(I) increasing or decreasing memory B cells, naïve B cells, and/or CD8+ T cells; and/or
(II) increasing or decreasing memory CD4+ T cells, resting CD4+ T cells, and/or naïve CD4+ T cells.
(III) increasing or decreasing innate immune cells, optionally wherein the innate immune cells are monocytes, macrophages, and/or dendritic cells.

17. The method according to any one of claims 1-16, further comprising detecting the expression or function of one or more of the genes the expression of which is to be increased or decreased, wherein said detecting occurs prior, during and/or after treatment.

18. The method of claim 17, wherein the expression or function of one or more of the genes is detected in one or more samples from the treated subject, optionally a blood sample, a skin sample, a vascular sample, a gastrointestinal tract sample, a lung sample, a heart sample, and/or a kidney sample.

19. A method of determining whether a therapy for a fibrotic, autoimmune, and/or inflammatory disease or condition is effective in a subject, comprising:
(a) measuring the expression of one or more genes listed in FIG. 2A in a sample from the subject before and at one or more time points after starting the therapy; and
(b) determining that the therapy is effective if:
(i) one or more genes listed in the second half of FIG. 2A (genes shown in FIG. 2A (continued)) are downregulated in at least one time point after starting the therapy compared to before starting the therapy; and/or
(ii) one or more genes listed in the first half of FIG. 2A are upregulated in at least one time point after starting the therapy compared to before starting the therapy,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
and further optionally wherein the fibrotic, autoimmune, and/or inflammatory disease or condition is SSc.

20. The method of claim 19, wherein the sample is a blood sample, a skin sample, a vascular sample, a gastrointestinal tract sample, a lung sample, a heart sample, and/or a kidney sample.

21. A method of determining whether a therapy for a fibrotic, autoimmune, and/or inflammatory disease or condition is effective in a subject, comprising:
(a) measuring the expression of one or more genes listed in FIG. 2A in a sample from the subject before and at one or more time points after starting the therapy, optionally wherein the one or more genes at least include ACVR1C;
(b) quantifying the numbers and/or percentage of memory B cells, naïve B cells, memory CD4+ T cells, resting CD4+ T cells, naïve CD4+ T cells, and/or CD8+ T cells in a sample from the subject before and at one or more time points after starting the therapy; and
(c) determining that the therapy is effective if:
(i) one or more genes listed in the second half of FIG. 2A (genes shown in FIG. 2A (continued)) are downregulated in at least one time point after starting the therapy compared to before starting the therapy; and/or
(ii) one or more genes listed in the first half of FIG. 2A are upregulated in at least one time point after starting the therapy compared to before starting the therapy,
and if:
(I) the number and/or percentage of memory B cells, naïve B cells, and/or CD8+ T cells is increased relative to a healthy control; and/or
(II) the number and/or percentage of memory CD4+ T cells, resting CD4+ T cells, and/or naïve CD4+ T cells, is decreased relative to a healthy control.

22. The method of claim 21, wherein the sample in (a) and/or (b) is a blood sample, a skin sample, a vascular sample, a gastrointestinal tract sample, a lung sample, a heart sample, and/or a kidney sample.

23. The method of any one of claims 19-22, wherein said one or more genes in (i) are ACVR1C, GREM2, NOG, ZFYVE9, NR3C2, LOC100131662, CFHR3, IL7R, and/or DNMT3A, and/or said one or more genes in (ii) are E2F5, CD19, IL6, ZNF204P, and/or PLEKHF2.

24. A method of screening for a therapeutic agent for a fibrotic, autoimmune, and/or inflammatory disease or condition, comprising:
(a) applying a candidate therapeutic agent to (I) one or more cells derived from a patient having the disease or condition, (II) one or more cell line cells of the disease or condition, or (III) a cell or tissue culture comprising a sample derived from a patient having the disease or condition;
(b) after step (a), measuring the expression of one or more genes listed in FIG. 2A in the (I) the one or more cells, (II) the one or more cell line cells, or (III) the cell or tissue culture;
(c) determining that a candidate therapeutic agent is effective if:
(i) one or more genes listed in the second half of FIG. 2A (genes shown in FIG. 2A (continued)) are downregulated compared to an untreated or placebo control; and/or
(ii) one or more genes listed in the first half of FIG. 2A are upregulated compared to an untreated or placebo control.

25. The method of claim 24, wherein said one or more genes in (i) are ACVR1C, GREM2, NOG, ZFYVE9, NR3C2, LOC100131662, CFHR3, IL7R, and/or DNMT3A, and/or said one or more genes in (ii) are E2F5, CD19, IL6, ZNF204P, and/or PLEKHF2.

26. The method of claim 24 or 25, wherein the one or more cells in (I) or (II) comprise a skin cell, a blood cell, an immune cell, a vascular cell, a gastrointestinal cell, a lung cell, a heart cell, and/or a renal cell, optionally wherein the one or more cells in (I) or (II) comprise an adipocyte, an epithelial cell, a fibroblast, a monocyte, a macrophage, a dendritic cell, a T cell, an endothelial cell, a muscle cell, and/or a fibrocyte, further optionally wherein the epithelial cell is a keratinocyte.

27. The method of any one of claim 24, 25 or 26, wherein the cell or tissue culture in (III) comprises a skin tissue, an organoid, or a three-dimensional layered cell culture.

28. The method of any one of claim 24, 25 or 26, wherein the sample derived from a patient having the disease or condition in (III) comprises:
(i) one or more fibroblasts derived from a patient having the disease or condition;
(ii) one or more monocytes/macrophages derived from a patient having the disease or condition; and/or
(iii) one or more keratinocytes derived from a patient having the disease or condition;
(iv) serum or plasma derived from a patient having the disease or condition.

29. The method of any one of claim 24, 25 or 26, wherein the cell or tissue culture in (III) is a three-dimensional, skin-like layered cell culture comprising:
(i) one or more fibroblasts; and
(ii) one or more keratinocytes;
and optionally
(iii) one or more monocytes or macrophages;
(iv) one or more T cells, and/or
(v) serum or plasma,
wherein at least one of (i)-(iv) is derived from a patient having the fibrotic, autoimmune, and/or inflammatory disease or condition.

30. A method of predicting whether a subject with a fibrotic, autoimmune, and/or inflammatory disease or condition will respond to HSCT, comprising:
(a) measuring the expression of ACVR1C in a sample from the subject; and
(b) determining that the subject will be a good responder to HSCT if the subject is a high expresser of ACVR1C,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
further optionally wherein the fibrotic, autoimmune, and/or inflammatory disease or condition is SSc,
and further optionally wherein the SSc is fibroproliferative SSc, normal-like SSc, or inflammatory SSc.

31. A method of treating a subject having a fibrotic, autoimmune, and/or inflammatory disease or condition, comprising:
(a) measuring the expression of ACVR1C in a sample from the subject; and
(b) treating the subject with hematopoietic stem cell transplant (HSCT) if the subject is a high expresser of ACVR1C,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
further optionally wherein the fibrotic, autoimmune, and/or inflammatory disease or condition is SSc,
and further optionally wherein the SSc is fibroproliferative SSc, normal-like SSc, or inflammatory SSc.

32. A method of predicting whether a subject with a fibrotic, autoimmune, and/or inflammatory disease or condition will respond to cyclophosphamide (CYC) treatment, comprising:
(a) measuring the expression of CLCF1 in a sample from the subject; and
(b) determining that the subject will be a good responder to CYC if the subject is a high expresser of CLCF1,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
further optionally wherein the fibrotic, autoimmune, and/or inflammatory disease or condition is SSc,
and further optionally wherein the SSc is fibroproliferative SSc, normal-like SSc, or inflammatory SSc.

33. A method of treating a subject having a fibrotic, autoimmune, and/or inflammatory disease or condition, comprising:
(a) measuring the expression of CLCF1 in a sample from the subject; and
(b) treating the subject with CYC if the subject is a high expresser of CLCF1 optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
further optionally wherein the fibrotic, autoimmune, and/or inflammatory disease or condition is SSc,
and further optionally wherein the SSc is fibroproliferative SSc, normal-like SSc, or inflammatory SSc.

34. The method according to any one of claims 30-33, wherein the sample is a blood sample, a skin sample, a vascular sample, a gastrointestinal tract sample, a lung sample, a heart sample, and/or a kidney sample.

35. An agent for treating or preventing a fibrotic, autoimmune, and/or inflammatory disease or condition, selected from:
(i) one which decreases the expression of one or more genes listed in the second half of FIG. 2A (genes shown in FIG. 2A (continued))
(ii) one which suppresses, blocks, or inhibits the function of the gene product of one or more genes listed in the second half of FIG. 2A (genes shown in FIG. 2A (continued));
(iii) one which decreases the expression or function of ACVR1C, GREM2, NOG, ZFYVE9, NR3C2, LOC100131662, CFHR3, IL7R, and/or DNMT3A;
(iv) one which increases the expression of one or more genes listed in the first half of FIG. 2A;
(v) one which enhances the function of the gene product of one or more genes listed in the second half of FIG. 2A (genes shown in FIG. 2A (continued));
(vi) one which enhances the expression or function of E2F5, CD19, IL6, ZNF204P, and/or PLEKHF2;
or any combination of the foregoing, further wherein the agent optionally is selected from an antibody, an antigen-binding antibody fragment (e.g., scFv, Fab, Fab′, (Fab′)₂), a chimeric antigen receptor (CAR)-expressing cell, an siRNA, an shRNA, an miRNA, an aptamer, a CRISPR/Cas-based gene therapy agent, a peptide, a small molecule, a polymer, an expression vector encoding a gene of interest, or any combination thereof,
optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
further optionally wherein the fibrotic, autoimmune, and/or inflammatory disease or condition is SSc.

36. The agent of claim 35 which comprises (i) a neutralizing, blocking, and/or antagonistic antibody or antigen-binding antibody fragment specific for ACVR1C, GREM2, NOG, ZFYVE9, NR3C2, LOC100131662, CFHR3, IL7R, and/or DNMT3A, (ii) a neutralizing, blocking, and/or antagonistic antibody or antigen-binding antibody fragment specific for ACVR1C, which optionally is designed to specifically or preferentially neutralize, block, and/or antagonize ACVR1C on macrophages, fibroblasts, and/or keratinocytes; (iii) an agonistic antibody or antigen-binding antibody fragment specific for E2F5, CD19, IL6, ZNF204P, and/or PLEKHF2; or (iv) any combination of any of the foregoing or a composition containing any of the foregoing.

37. A kit comprising:
(a) at least one primer set for detecting expression of at least one gene listed in FIG. 2A; and
(b) an instruction sheet.

38. A kit comprising:
(a) (I) one or more cells derived from a patient with a fibrotic, autoimmune, and/or inflammatory disease or condition, (II) one or more cell line cells of a fibrotic, autoimmune, and/or inflammatory disease or condition, or (III) a cell or tissue culture comprising a sample derived from a patient with a fibrotic, autoimmune, and/or inflammatory disease or condition; and
(b) at least one primer set for detecting expression of at least one gene listed in FIG. 2A,
optionally wherein the kit is for screening a therapeutic agent for treating a fibrotic, autoimmune, and/or inflammatory disease or condition,
further optionally wherein the fibrotic, autoimmune, and/or inflammatory disease or condition is systemic sclerosis (SSc), keloid, nephrogenic systemic fibrosis, interstitial lung disease (ILD), pulmonary fibrosis (PF) (with or without association to an autoimmune disease), idiopathic pulmonary fibrosis (IPF), rheumatoid arthritis (RA)-associated interstitial lung disease (RA-ILD), cystic fibrosis (CF), asthma, chronic obstructive pulmonary disease (COPD), chronic lupus pneumonititis, hepatic fibrosis (e.g., bridging fibrosis), radiation-induced lung injury (e.g., upon radiation therapy progressive massive fibrosis, for cancer), inflammatory bowel disease (IBD), ulcerative colitis (UC), Crohn's disease (CD), myocardial fibrosis (e.g., interstitial fibrosis or replacement fibrosis), mediastinal fibrosis, retroperitoneal fibrosis, myelofibrosis, arterial stiffness, arthrofibrosis, Dupuytren's contracture, Peyronie's disease, adhesive capsulitis, multiple sclerosis (MS), systemic lupus erythematosus (SLE), type 1 diabetes (T1D), rheumatoid arthritis (RA), reactive arthritis, Celiac disease, vasculitis, Grave's disease, Hashimoto thyroiditis, myasthenia gravis (MG), psoriasis, dermatomyositis, Adison's disease, Sjogren syndrome, Guillain-Barre syndrome, or Chronic inflammatory demyelinating polyneuropathy (CIDP),
and further optionally wherein the fibrotic, autoimmune, and/or inflammatory disease or condition is SSc.

39. The kit according to claim 37 or 38, wherein the at least one gene comprises ACVR1C.


SEQUENCE LISTING (4 sequences)

SEQ ID NO: 1 - protein, 493 aa, Artificial Sequence
  isoforms due to alternative splicing
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 2 - protein, 443 aa, Artificial Sequencing
  isoforms due to alternative splicing
MLTNGKEQVIKSCVSLPELNAQVFCHSSNNVTKTECCFTDFCNNITLHLPTASPNAPKLGPMELAIIITVPVCLLSIAAMLTVWACQGRQCSYRKKKRPNVEEPLSECNLVNAGKTLKDLIYDVTASGSGSGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 3 - protein, 413 aa, Artificial Sequence
  isoforms due to alternative splicing
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTGLPLLVQRTIARTIVLQEIVGKGRFGEVWHGRWCGEDVAVKIFSSRDERSWFREAEIYQTVMLRHENILGFIAADNKDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA

SEQ ID NO: 4 - protein, 336 aa, Artificial Sequence
  isoforms due to alternative splicing
MTRALCSALRQALLLLAAAAELSPGLKCVCLLCDSSNFTCQTEGACWASVMLTNGKEQVIKSCVSLPELNAQVFCHSSNNVTKTECCFTDFCNNITLHLPTDNGTWTQLWLVSEYHEQGSLYDYLNRNIVTVAGMIKLALSIASGLAHLHMEIVGTQGKPAIAHRDIKSKNILVKKCETCAIADLGLAVKHDSILNTIDIPQNPKVGTKRYMAPEMLDDTMNVNIFESFKRADIYSVGLVYWEIARRCSVGGIVEEYQLPYYDMVPSDPSIEEMRKVVCDQKFRPSIPNQWQSCEALRVMGRIMRECWYANGAARLTALRIKKTISQLCVKEDCKA